(19) United States
(12) Patent Application Publication
(10) Pub. No.: US 2019/0154670 A1
(43) Pub. Date: May 23, 2019

(54) SWCNT-DNA-ANTIBODY CONJUGATES, RELATED COMPOSITIONS, AND SYSTEMS, METHODS AND DEVICES FOR THEIR USE

(71) Applicant: Memorial Sloan Kettering Cancer Center, New York, NY (US)

(72) Inventors: Daniel A. HELLER, New York, NY (US); Ryan M. WILLIAMS, New York, NY (US)

(73) Assignee: Memorial Sloan Kettering Cancer Center, New York, NY (US)

(21) Appl. No.: 16/099,662

(22) PCT Filed: Apr. 7, 2017
(86) PCT No.: PCT/US2017/026563

§ 371 Date: Nov. 7, 2018

Related U.S. Application Data
(60) Provisional application No. 62/334,412, filed on May 10, 2016.

(51) Int. Cl. G01N 33/543; G01N 33/551; A61B 5/1459; A61B 5/1473

(52) U.S. Cl. CPC G01N 33/5438; G01N 33/551; G01N 33/54353; A61B 2562/242; A61B 5/14735; A61B 2562/0285; A61B 5/1459

(57) ABSTRACT

Described herein are compositions useful for the detection of analytes. In certain embodiments, the invention relates among other things to DNA-encapsulated single -walled carbon nanotubes (SWCNTs) functionalized with an antibody or other analyte-binding species, for detection and/or imaging of an analyte in a biological sample or subject. Other embodiments described herein include systems, methods, and devices utilizing such compositions for ex vivo biomarker quantification, tissue optical probes, and in vivo analyte detection and quantification. In one aspect the invention relates to a single -walled carbon nanotube (SWCNT) sensor, comprising a SWCNT; a polymer associated with the SWCNT; and an analyte-binding species. Detection of one or more analytes is achieved by measuring changes in fluorescence intensity, shifts in fluorescence wavelength, and/or other characteristics in the spectral characteristics of the described compositions.

### CROSS REFERENCE TO RELATED APPLICATIONS

This application claims the benefit of U.S. Application Ser. No. 62/334,412 filed on May 10, 2016, the disclosure of which is hereby incorporate by reference in its entirety. Applicant also notes it is concurrently filing a potentially related patent application entitled, “SENSORS FOR NUCLEIC ACID BIOMARKERS”, which claims the benefit of U.S. Application Ser. No. 62/320,126 filed on Apr. 8, 2016.

### GOVERNMENT FUNDING

This invention was made with government support under grant numbers HD075698 and CA008748 awarded by National Institutes of Health. The government has certain rights in this invention.

### FIELD OF INVENTION

This invention relates generally to compositions, systems, methods, and devices for the detection of analytes. In certain embodiments, the invention relates to DNA-encapsulated single-walled carbon nanotubes (SWCNTs) functionalized with an antibody or other analyte-binding species, for detection and/or imaging of an analyte in a biological sample or subject.

### BACKGROUND

Current techniques for detection of protein biomarkers for the diagnosis and monitoring of diseases lack the sensitivity and specificity necessary for widespread clinical use. For example, worldwide, over 238,000 patients are diagnosed with ovarian cancer, a disease which is responsible for over 151,000 deaths each year. Ovarian cancer is the fifth-leading cause of cancer-related deaths among females in the United States and first among gynecologic malignancies. These grim statistics are due in part to the advanced stage at which most cases are detected—at stage III or later in more than 60% of diagnoses—higher than any other form of cancer. Among all populations, the five-year survival rate is just 46%. In cases where diagnosis occurs at stage I, however, the five-year survival is 92%. Existing screening methods include CA125 serum testing and transvaginal ultrasonography, for which the USPSTF recommends against use due to a high false-positive rate and poor sensitivity for detecting small lesions. These methods neither alter patient outcome nor reduce mortality.

Human epididymis protein 4 (HE4) is one of two FDA-approved serum biomarkers for ovarian cancer, along with CA125, and it may play a factor in ovarian tumorigenesis. This protein is overexpressed by malignant epithelial cells and found in increased levels in patient serum, ascites, and uterine fluid. HE4 provides similar sensitivity and specificity for ovarian cancer diagnosis as CA125, although it may be more useful in differentiating benign from malignant disease. However, like CA125, data does not show that serum-based screening for HE4 improves overall patient survival.

Single-walled carbon nanotubes (SWCNT) have electronic and optical properties that are well-suited for in vivo signal transduction. Semiconducting carbon nanotubes emit near-infrared (NIR) bandgap photoluminescence between 800 and 1600 nm which can penetrate living tissues to a distance in the centimeter range. Carbon nanotubes have been investigated for use in vivo to image vasculature and as intraoperative probes. Carbon nanotube fluorescence exhibits unique photostability, allowing for repeated, long-term measurements. Nanotubes also exhibit exquisite sensitivity to their local environment via optical bandgap modulation. Prior works have demonstrated the optical detection of small molecules, and proteins in/on live cells and to measure reactive oxygen species in living organisms. However, such works have not successfully detected cancer biomarkers (e.g., ovarian cancer biomarkers) with the sensitivity and specificity required for clinical use and implementation. Further, such works have not utilized the optical properties of SWCT to detect cancer protein biomarkers (e.g., ovarian cancer biomarkers).

Therefore, new methods are needed to detect diseases, such as early-stage cancer (e.g., ovarian cancer), in order to reduce the burden of diseases (e.g., ovarian cancer).

### SUMMARY OF INVENTION

Described herein are compositions useful for the detection of analytes. In certain embodiments, the invention relates to DNA-encapsulated single-walled carbon nanotubes (SWCNTs) functionalized with an antibody or other analyte-binding species, for detection and/or imaging of an analyte in a biological sample or subject. Other embodiments described herein include systems, methods, and devices utilizing such compositions for ex vivo biomarker quantification, tissue optical probes, and in vivo analyte detection and quantification.

In certain embodiments, single-stranded DNA-encapsulated single-walled carbon nanotubes (SWCNTs) are functionalized with an antibody (species). The synthesis preserves the optical properties of carbon nanotubes and can be used for applications including optical sensors for ex vivo biomarker quantification, optical probes for tissue labeling, and in vivo analyte detection and quantification, for example.

In certain embodiments, to synthesize the sensor complex, SWCNTs are ultrasonicated with polymeric materials such as oligmeric ssDNA that is functionalized with a primary amine. The amine is used to chemically conjugate SWCNT to an antibody specific for the desired analyte through an amidation reaction. Upon binding of the biomarker to the antibody, a specific change in the intrinsic fluorescence of the SWCNT is observed and measured by fluorescence microscopy, spectroscopy, or a portable light detector. This device can be used for the specific and rapid detection of biomarkers in patient samples such as whole blood, serum, urine, and the like.

Such complexes can be used in a variety of biosensing applications. For example, cancer biomarkers or other analytes may be identified, imaged (mapped), and/or quantified in research applications or patient samples ex vivo. Biomarker concentrations can be accurately and specifically determined in a sample much more rapidly than current assays. This allows for use of the sensor as a quantification tool in research applications, akin, for example, to an enzyme-linked immunosorbent assay (ELISA) or Western blot. The sensor can also perform the same function in patient samples such as blood, serum, plasma, urine, ascites, and uterine washings.

Such complexes can also be used for the fluorescent labeling of multiple biomarkers or antigens in a tissue slice or cell culture from animals or human patients. A need exists for fluorescent tissue labeling of many analytes within a single tissue section. Current fluorescent molecules conjugated to antibodies that label analytes within a tissue section can only be multiplexed with a total of approximately 5 detection antibodies. With individual carbon nanotube species, greater than 12 spectrally distinct fluorescent labels are possible. Use of complexes described herein significantly increases the multiplexed capability of tissue analyte localization and quantification.

Devices with the complexes described herein can also be used in vivo for specific detection of circulating or localized biomarkers or tumor cells in a living organism. In this mode, the sensor complex can be immobilized in a biocompatible gel, microcapillary, filter, mesh, tubing, or other solid support. This biosensing device can be implanted in vivo subcutaneously, intraperitoneally, within the venous system, including the inferior vena cava. The sensor can be interrogated from outside the body using a light source that can be directed at the device using near-infrared excitation light, or from within a body cavity using a minimally-invasive procedure. The light emitted from the nanotubes in the device can penetrate tissue up to a distance of several centimeters in order to collect the light using a detector which may be stationary or portable. Thus, such embodiments address the need for rapid, transient, specific, and sensitive detection of disease biomarkers in a patient. This allows for more informed decisions by the patient and physician about therapies/courses of treatment.

For example, the carbon nanotube-based sensors described herein can non-invasively detect the ovarian cancer biomarker HE4 in vivo. As described herein, for example, the sensor can be developed by derivatizing NIR-emitting carbon nanotubes to transduce the binding of HE4 to an immobilized antibody. The antibody-nanotube complex responds specifically to HE4 via modulation of the nanotube emission wavelength. Further, quantitative responses differentiated HGSC from control patient serum and ascites samples. In addition to in vivo measurements, an implantable device incorporating the antibody-nanotube complex was engineered and probed non-invasively via NIR optical excitation and collection from the live mouse. The device successfully quantified HE4 in four orthotopic murine models of ovarian cancer. Other biomarkers that are applicable to a variety of diseases can also be detected using the carbon-nanotube-based sensors.

Techniques described herein can be used for in vivo, ex vivo, and/or in vitro detection of protein biomarkers for diagnosis and/or monitoring of diseases or conditions (e.g., cancers, e.g., acute kidney disease, e.g., heart disease, e.g., vascular disease, e.g., diabetes, e.g., infection diseases).

In one aspect, the invention is directed to a single-walled carbon nanotube (SWCNT) sensor, comprising a SWCNT; a polymer associated with the SWCNT (e.g., conjugated non-covalently or covalently to the SWCNT (e.g., directly or via a linker) and/or wrapped around (e.g., encapsulating) the SWCNT); and an analyte-binding species.

In certain embodiments, the analyte-binding species comprises a member selected from the group consisting of a peptide, polypeptide, and a protein. In certain embodiments, the analyte-binding species comprises an antibody. In certain embodiments, the analyte-binding species is attached to the polymer (e.g., single-stranded DNA) via a functional group (e.g., an amine, e.g., primary amine). In certain embodiments, the analyte-binding species binds a desired analyte; whereupon binding of the analyte to the analyte-binding species results in a detectable change in intrinsic fluorescence of the SWCNT.

In certain embodiments, the desired analyte comprises a member selected from the group consisting of HE4, CA-125, mesothelin, CRABP2 (cellular retinoic acid binding protein 2), and YKL-4. In certain embodiments, the desired analyte comprises a member selected from the group consisting of uPAR (uPA receptor), CA125, CRABP2 (cellular retinoic acid binding protein 2), mesothelin, YKL-40, PSA (prostate specific antigen), PSMA (prostate specific membrane antigen), carcinoembryonic antigen (CEA), and MUC1.

In certain embodiments, the linker comprises a member selected from the group consisting of a 6 carbon (C6) linker, polyethylene glycol (PEG), a hydrocarbon, a synthetic polymer, and a biopolymer (e.g., a DNA, e.g., RNA, e.g., polypeptide).

In certain embodiments, the polymer comprises a member selected from the group consisting of DNA, LNA, PNA, an amino-acid sequence, and a synthetic monomer. In certain embodiments, the polymer comprises DNA, and wherein the DNA is single-stranded DNA.

In another aspect, the invention is directed to a device for ex vivo detection of one or more analytes of interest in a biological sample, the device comprising a SWCNT; a polymer associated with the SWCNT; and an analyte-binding species.

In certain embodiments, the biological sample comprises a member selected from the group consisting of blood, serum, plasma, urine, ascites, and uterine washing. In certain embodiments, the biological sample comprises a tissue sample or cell culture.

In another aspect, the invention is directed to a device for detection of multiple analytes of interest in a biological sample, the device comprising one or more single-walled carbon nanotube (SWCNT) sensors, each of the one or more SWCNT comprising: a SWCNT; a polymer associated with the SWCNT; and an analyte-binding species that, collectively, comprise a plurality of (species of) detection analyte-binding species each of which identify a different analyte of interest.

In certain embodiments, the plurality of detection analyte-binding species comprise a plurality of antibodies (e.g., at least 5, e.g., at least 7, e.g., at least 9, e.g., at least 10, e.g., at least 11, e.g., at least 12). In certain embodiments, each species of analyte-binding species binds a desired analyte; whereupon binding of the analyte to a particular analyte-binding species results in a detectable change in intrinsic fluorescence of the SWCNT which is distinguishable from the detectable change in intrinsic fluorescence of the SWCNT resulting from the binding of any of the other analytes to any of the other analyte-binding species present in the device.

In certain embodiments, the biological sample comprises a member selected from the group consisting of tissue, cell culture, blood, serum, plasma, urine, ascites, and uterine washing.

In another aspect, the invention is directed to a system comprising the device, the system further comprising an excitation light source (e.g., near-infrared light) and a detector for detecting light (e.g., fluorescent light) emitted from nanotubes in the device following excitation by the excitation light source.

In certain embodiments, whereupon binding of the analyte to the antibody results in a detectable change in intrinsic fluorescence of the SWCNT.

In another aspect, the invention is directed to an implantable device comprising one or more single-walled carbon nanotube (SWCNT) sensors, each of the one of more SWCNT sensors comprising: a SWCNT; a polymer associated with the SWCNT; and an analyte-binding species.

In certain embodiments, the device further comprises a solid support by which the device is immobilized (e.g., wherein the solid support comprises a biocompatible gel, microcapillary, filter, mesh, tubing, compartment/dialysis membrane, or other solid support).

In certain embodiments, the device is a point-of-care medical device (e.g., a urine dipstick, a test strip, a membrane, a skin patch, a skin probe, a gastric band, a stent, a catheter, a needle, a contact lens, a prosthetic, a denture, a vaginal ring, or other implant). In certain embodiments, the device comprises a microfluidic chamber containing a surface-immobilized SWCNT sensor, or an SWCNT sensor contained in a semi-permeable enclosure.

In another aspect, the invention is directed to a system for in vivo detection of (e.g., circulating and/or localized) biomarkers and/or tumor cells in a subject, the system comprising: an implantable device comprising one or more single-walled carbon nanotube (SWCNT) sensors, each of the one or more SWCNT comprising a SWCNT, a polymer associated with the SWCNT, and an analyte-binding species; an excitation light source; and a detector for detecting light emitted from the nanotubes in the (implanted) implantable device following excitation by the excitation light source.

In certain embodiments, the device comprises a biocompatible gel, microcapillary, filter, mesh, tubing, compartment/dialysis membrane, and/or other solid support on or in which the sensor is immobilized.

In certain embodiments, the excitation light source emits near-infrared light or light having a wavelength greater than 700 nm. In certain embodiments, the detector detects fluorescent light.

In certain embodiments, the implantable device is shaped and sized for implantation subcutaneously, intraperitoneally, and/or within the venous system. In certain embodiments, the implantable device is shaped and sized to be an intrauterine device (IUD) (e.g., about 20 to 40 mm in width, e.g., about 20 to 40 mm in length). In certain embodiments, the implantable device is shaped and sized so that the device is delivered via injection (e.g., via a syringe). In certain embodiments, the implantable device is shaped and sized for implantation in the inferior vena cava. In certain embodiments, the implantable device is configured for (sized, shaped, constructed for) attachment to or embedding (partially or wholly) within a wall of a body cavity, lumen, or organ. In certain embodiments, the body cavity, lumen, or organ comprises a member selected from the group consisting of uterine cavity, cranial cavity, vertebral canal, thoracic cavity, abdominal cavity, pelvic cavity, artery, vein, gastrointestinal tract, bronchi, renal tubules, urinary collecting ducts, vagina, uterus, fallopian tubes, adrenal gland, bone, esophagus, heart, larynx, mouth, pituitary gland, muscle, spleen, thyroid, anus, brain, eye, hypothalamus, liver, nose, prostate, skin, stomach, ureter, appendix, gall bladder, kidney, lung, pancreas, rectum, small intestine, thymus, urethra, bladder, ear, genitals, large intestine, lymph node, parathyroid gland, salivary gland, spinal cord, and trachea.

In certain embodiments, the excitation light source is positioned outside the subject (or within a body cavity of the subject via a minimally-invasive procedure) when transmitting excitation light through tissue of the subject to the implanted device comprising the one or more SWCNT sensors.

In certain embodiments, the detector and the excitation light source are part of the same unit. In certain embodiments, the unit comprises a handheld unit positioned outside the subject or within a body cavity of the subject.

In another aspect, the invention is directed to a method of detecting an analyte of interest (e.g., one or more analytes of interest) in a biological sample (or in the subject) using the sensor, device, or system of any one of the preceding claims, the method comprising: detecting a wavelength shift (e.g., a blueshift or a redshift) in emission EMR and/or an intensity shift (e.g., amplitude shift) and/or another change in the spectral characteristics of emission EMR, whereupon binding of the analyte to a particular analyte-binding species (e.g., antibody) results in a detectable change in the emission EMR (e.g., intrinsic fluorescence of the SWCNT), thereby identifying the presence of the analyte.

In certain embodiments, the method comprises identifying a concentration of the analyte of interest in the biological sample (or in the subject).

In certain embodiments, the method comprises rendering a 2D or 3D map of analyte presence or concentration in the biological sample or the subject.

In certain embodiments, the analyte of interest comprises one or more members selected from the group consisting of a peptide, a polypeptide, a protein, a biologic, a biomolecule, a biosimilar, an aptamer, a virus, a drug, a lipid, a bacterium, a toxin, a cell, a tumor cell, cancer, an antibody, and an antibody fragment. In certain embodiments, the analyte of interest is an ovarian cancer biomarker. In certain embodiments, the desired analyte comprises a member selected from the group consisting of HE4, CA-125, mesothelin, CRABP2 (cellular retinoic acid binding protein 2), and YKL-4. In certain embodiments, the desired analyte comprises a member selected from the group consisting of uPAR (uPA receptor), CA125, CRABP2 for ovarian cancer (cellular retinoic acid binding protein 2), mesothelin, YKL-40, PSA (prostate specific antigen), PSMA (prostate specific membrane antigen), carcinoembryonic antigen (CEA), and MUC1.

In certain embodiments, the biological sample is in vitro, ex vivo, or in vivo (e.g., wherein the biological sample is the subject). In certain embodiments, the biological sample comprises a member selected from the group consisting of a cell culture sample, a laboratory sample, a tissue sample (e.g., muscle tissue, nervous tissue, connective tissue, and epithelial tissue), and a bodily fluid sample. In certain embodiments, the bodily fluid sample comprises a member selected from the group consisting of Amniotic fluid, Aqueous humour and vitreous humour, Bile, Blood serum, Breast milk, Cerebrospinal fluid, Cerumen (earwax), Chyle, Chyme, Endolymph and perilymph, Exudates, Feces, Female ejaculate, Gastric acid, Gastric juice, Lymph, Menstrual fluid, Mucus (including nasal drainage and phlegm), Pericardial fluid, Peritoneal fluid, Pleural fluid, Pus, Rheum, Saliva, Sebum (skin oil), Serous fluid, Semen, Smegma, Sputum, Synovial fluid, Sweat, Tears, Urine, Uterine Washing, Vaginal secretion, and Vomit. In certain embodiments, the bodily fluid sample comprises serum. In certain embodiments, the bodily fluid comprises uterine washing. In certain embodiments, the bodily fluid comprises ascitic fluid (e.g., ascites). In certain embodiments, the bodily fluid comprises urine.

In another aspect, the invention is directed to a kit for use in a laboratory setting, the kit comprising: at least one container (e.g., an ampule, a vial, a cartridge, a reservoir, a lyoject, or a pre-filled syringe); and a single-walled carbon nanotube (SWCNT) sensor comprising: a SWCNT; a polymer associated with the SWCNT; and an analyte-binding species.

Elements of the embodiments involving one aspect of the invention (e.g., compositions) can be applied in embodiments involving one or more other aspects of the invention (e.g., systems, methods, and/or devices).

### Definitions

In order for the present disclosure to be more readily understood, certain terms are first defined below. Additional definitions for the following terms and other terms are set forth throughout the specification.

In this application, the use of “or” means “and/or” unless stated otherwise. As used in this application, the term “comprise” and variations of the term, such as “comprising” and “comprises,” are not intended to exclude other additives, components, integers or steps. As used in this application, the terms “about” and “approximately” are used as equivalents. Any numerals used in this application with or without about/approximately are meant to cover any normal fluctuations appreciated by one of ordinary skill in the relevant art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

“Administration:” The term “administration” refers to introducing a substance into a subject. In general, any route of administration may be utilized including, for example, parenteral (e.g., intravenous), oral, topical, subcutaneous, peritoneal, intraarterial, inhalation, vaginal, rectal, nasal, introduction into the cerebrospinal fluid, or instillation into body compartments. In some embodiments, administration is oral. Additionally or alternatively, in some embodiments, administration is parenteral. In some embodiments, administration is intravenous.

“Antibody”: As used herein, the term “antibody” refers to a polypeptide that includes canonical immunoglobulin sequence elements sufficient to confer specific binding to a particular target antigen. Intact antibodies as produced in nature are approximately 150 kD tetrameric agents comprised of two identical heavy chain polypeptides (about 50 kD each) and two identical light chain polypeptides (about 25 kD each) that associate with each other into what is commonly referred to as a “Y-shaped” structure. Each heavy chain is comprised of at least four domains (each about 110 amino acids long)—an amino-terminal variable (VH) domain (located at the tips of the Y structure), followed by three constant domains: CH₁, CH₂, and the carboxy-terminal CH₃ (located at the base of the Y's stem). A short region, known as the “switch”, connects the heavy chain variable and constant regions. The “hinge” connects CH₂ and CH₃ domains to the rest of the antibody. Two disulfide bonds in this hinge region connect the two heavy chain polypeptides to one another in an intact antibody. Each light chain is comprised of two domains—an amino-terminal variable (VL) domain, followed by a carboxy-terminal constant (CL) domain, separated from one another by another “switch”. Intact antibody tetramers are comprised of two heavy chain-light chain dimers in which the heavy and light chains are linked to one another by a single disulfide bond; two other disulfide bonds connect the heavy chain hinge regions to one another, so that the dimers are connected to one another and the tetramer is formed. Naturally-produced antibodies are also glycosylated, typically on the CH₂ domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops known as “complement determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. The Fc region of naturally-occurring antibodies binds to elements of the complement system, and also to receptors on effector cells, including for example effector cells that mediate cytotoxicity. Affinity and/or other binding attributes of Fc regions for Fc receptors can be modulated through glycosylation or other modification. In certain embodiments, antibodies produced and/or utilized in accordance with the present invention include glycosylated Fc domains, including Fc domains with modified or engineered such glycosylation. For purposes of the present invention, in certain embodiments, any polypeptide or complex of polypeptides that includes sufficient immunoglobulin domain sequences as found in natural antibodies can be referred to and/or used as an “antibody”, whether such polypeptide is naturally produced (e.g., generated by an organism reacting to an antigen), or produced by recombinant engineering, chemical synthesis, or other artificial system or methodology. In certain embodiments, an antibody is polyclonal; in certain embodiments, an antibody is monoclonal. In certain embodiments, an antibody has constant region sequences that are characteristic of mouse, rabbit, primate, or human antibodies. In certain embodiments, antibody sequence elements are humanized, primatized, chimeric, etc., as is known in the art. Moreover, the term “antibody” as used herein, can refer in appropriate embodiments (unless otherwise stated or clear from context) to any of the art-known or developed constructs or formats for utilizing antibody structural and functional features in alternative presentation. For example, embodiments, an antibody utilized in accordance with the present invention is in a format selected from, but not limited to, intact IgG, IgE and IgM, bi- or multi-specific antibodies (e.g., Zybodies®, etc), single chain Fvs, polypeptide-Fc fusions, Fabs, cameloid antibodies, masked antibodies (e.g., Probodies®), Small Modular ImmunoPharmaceuticals (“SMIPs™”), single chain or Tandem diabodies (TandAb®), VHHs Anticalins®, Nanobodies®, minibodies, BiTE®s, ankyrin repeat proteins or DARPINs®, Avimers®, a DART, a TCR-like antibody, Adnectins®, Affilins®, Trans-bodies®, Affibodies®, a TrimerX®, MicroProteins, Fynomers®, Centyrins®, and a KALBITOR®. In certain embodiments, an antibody may lack a covalent modification (e.g., attachment of a glycan) that it would have if produced naturally. In certain embodiments, an antibody may contain a covalent modification (e.g., attachment of a glycan, a payload [e.g., a detectable moiety, a therapeutic moiety, a catalytic moiety, etc], or other pendant group [e.g., poly-ethylene glycol, etc.]).

“Antibody fragment”: As used herein, an “antibody fragment” includes a portion of an intact antibody, such as, for example, the antigen-binding or variable region of an antibody. Examples of antibody fragments include Fab, Fab′, F(ab′)2, and Fv fragments; triabodies; tetrabodies; linear antibodies; single-chain antibody molecules; and multi specific antibodies formed from antibody fragments. For example, antibody fragments include isolated fragments, “Fv” fragments, consisting of the variable regions of the heavy and light chains, recombinant single chain polypeptide molecules in which light and heavy chain variable regions are connected by a peptide linker (“ScFv proteins”), and minimal recognition units consisting of the amino acid residues that mimic the hypervariable region. In many embodiments, an antibody fragment contains sufficient sequence of the parent antibody of which it is a fragment that it binds to the same antigen as does the parent antibody; in certain embodiments, a fragment binds to the antigen with a comparable affinity to that of the parent antibody and/or competes with the parent antibody for binding to the antigen. Examples of antigen binding fragments of an antibody include, but are not limited to, Fab fragment, Fab′ fragment, F(ab′)2 fragment, scFv fragment, Fv fragment, dsFv diabody, dAb fragment, Fd′ fragment, Fd fragment, and an isolated complementarity determining region (CDR) region. An antigen binding fragment of an antibody may be produced by any means. For example, an antigen binding fragment of an antibody may be enzymatically or chemically produced by fragmentation of an intact antibody and/or it may be recombinantly produced from a gene encoding the partial antibody sequence. Alternatively or additionally, antigen binding fragment of an antibody may be wholly or partially synthetically produced. An antigen binding fragment of an antibody may optionally comprise a single chain antibody fragment. Alternatively or additionally, an antigen binding fragment of an antibody may comprise multiple chains which are linked together, for example, by disulfide linkages. An antigen binding fragment of an antibody may optionally comprise a multimolecular complex. A functional single domain antibody fragment is in a range from about 5 kDa to about 25 kDa, e.g., from about 10 kDa to about 20 kDa, e.g., about 15 kDa; a functional single-chain fragment is from about 10 kDa to about 50 kDa, e.g., from about 20 kDa to about 45 kDa, e.g., from about 25 kDa to about 30 kDa; and a functional fab fragment is from about 40 kDa to about 80 kDa, e.g., from about 50 kDa to about 70 kDa, e.g., about 60 kDa.

“Associated”: As used herein, the term “associated” typically refers to two or more entities in physical proximity with one another, either directly or indirectly (e.g., via one or more additional entities that serve as a linking agent), to form a structure that is sufficiently stable so that the entities remain in physical proximity under relevant conditions, e.g., physiological conditions. In some embodiments, associated moieties are covalently linked to one another. In some embodiments, associated entities are non-covalently linked. In some embodiments, associated entities are linked to one another by specific non-covalent interactions (e.g., by interactions between interacting ligands that discriminate between their interaction partner and other entities present in the context of use, such as, for example. streptavidin/avidin interactions, antibody/antigen interactions, etc.). Alternatively or additionally, a sufficient number of weaker non-covalent interactions can provide sufficient stability for moieties to remain associated. Exemplary non-covalent interactions include, but are not limited to, electrostatic interactions, hydrogen bonding, affinity, metal coordination, physical adsorption, host-guest interactions, hydrophobic interactions, pi stacking interactions, van der Waals interactions, magnetic interactions, electrostatic interactions, dipole-dipole interactions, etc.

“Cancer”: As used herein, the term “cancer” refers to a disease, disorder, or condition in which cells exhibit relatively abnormal, uncontrolled, and/or autonomous growth, so that they display an abnormally elevated proliferation rate and/or aberrant growth phenotype characterized by a significant loss of control of cell proliferation. In certain embodiments, a cancer may be characterized by one or more tumors. Those skilled in the art are aware of a variety of types of cancer including, for example, adrenocortical carcinoma, astrocytoma, basal cell carcinoma, carcinoid, cardiac, cholangiocarcinoma, chordoma, chronic myeloproliferative neoplasms, craniopharyngioma, ductal carcinoma in situ, ependymoma, intraocular melanoma, gastrointestinal carcinoid tumor, gastrointestinal stromal tumor (GIST), gestational trophoblastic disease, glioma, histiocytosis, leukemia (e.g., acute lymphoblastic leukemia (ALL), acute myeloid leukemia (AML), chronic lymphocytic leukemia (CLL), chronic myelogenous leukemia (CML), hairy cell leukemia, myelogenous leukemia, myeloid leukemia), lymphoma (e.g., Burkitt lymphoma [non-Hodgkin lymphoma], cutaneous T-cell lymphoma, Hodgkin lymphoma, mycosis fungoides, Sezary syndrome, AIDS-related lymphoma, follicular lymphoma, diffuse large B-cell lymphoma), melanoma, merkel cell carcinoma, mesothelioma, myeloma (e.g., multiple myeloma), myelodysplastic syndrome, papillomatosis, paraganglioma, pheochromacytoma, pleuropulmonary blastoma, retinoblastoma, sarcoma (e.g., Ewing sarcoma, Kaposi sarcoma, osteosarcoma, rhabdomyosarcoma, uterine sarcoma, vascular sarcoma), Wilms' tumor, and/or cancer of the adrenal cortex, anus, appendix, bile duct, bladder, bone, brain, breast, bronchus, central nervous system, cervix, colon, endometrium, esophagus, eye, fallopian tube, gall bladder, gastrointestinal tract, germ cell, head and neck, heart, intestine, kidney (e.g., Wilms' tumor), larynx, liver, lung (e.g., non-small cell lung cancer, small cell lung cancer), mouth, nasal cavity, oral cavity, ovary, pancreas, rectum, skin, stomach, testes, throat, thyroid, penis, pharynx, peritoneum, pituitary, prostate, rectum, salivary gland, ureter, urethra, uterus, vagina, or vulva.

“Nucleic acid:” as used herein, in its broadest sense, refers to any compound and/or substance that is or can be incorporated into an oligonucleotide chain. In some embodiments, a nucleic acid is a compound and/or substance that is or can be incorporated into an oligonucleotide chain via a phosphodiester linkage. As will be clear from context, in some embodiments, “nucleic acid” refers to individual nucleic acid residues (e.g., nucleotides and/or nucleosides); in some embodiments, “nucleic acid” refers to an oligonucleotide chain comprising individual nucleic acid residues. In some embodiments, a “nucleic acid” is or comprises RNA; in some embodiments, a “nucleic acid” is or comprises DNA. In some embodiments, a nucleic acid is, comprises, or consists of one or more natural nucleic acid residues. In some embodiments, a nucleic acid is, comprises, or consists of one or more nucleic acid analogs. In some embodiments, a nucleic acid analog differs from a nucleic acid in that it does not utilize a phosphodiester backbone. For example, in some embodiments, a nucleic acid is, comprises, or consists of one or more “peptide nucleic acids”, which are known in the art and have peptide bonds instead of phosphodiester bonds in the backbone, are considered within the scope of the present invention. Alternatively or additionally, in some embodiments, a nucleic acid has one or more phosphorothioate and/or 5′-N-phosphoramidite linkages rather than phosphodiester bonds. In some embodiments, a nucleic acid is, comprises, or consists of one or more natural nucleosides (e.g., adenosine, thymidine, guanosine, cytidine, uridine, deoxyadenosine, deoxythymidine, deoxy guanosine, and deoxycytidine). In some embodiments, a nucleic acid is, comprises, or consists of one or more nucleoside analogs (e.g., 2-aminoadenosine, 2-thiothymidine, inosine, pyrrolo-pyrimidine, 3 -methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5 -propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof). In some embodiments, a nucleic acid comprises one or more modified sugars (e.g., 2′-fluororibose, ribose, 2′-deoxyribose, arabinose, and hexose) as compared with those in natural nucleic acids. In some embodiments, a nucleic acid has a nucleotide sequence that encodes a functional gene product such as an RNA or protein. In some embodiments, a nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 1 10, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, a nucleic acid is single stranded; in some embodiments, a nucleic acid is double stranded. In some embodiments a nucleic acid has a nucleotide sequence comprising at least one element that encodes, or is the complement of a sequence that encodes, a polypeptide. In some embodiments, a nucleic acid has enzymatic activity.

“Peptide” or “Polypeptide”: The term “peptide” or “polypeptide” refers to a string of at least two (e.g., at least three) amino acids linked together by peptide bonds. In certain embodiments, a polypeptide comprises naturally-occurring amino acids; alternatively or additionally, in certain embodiments, a polypeptide comprises one or more non-natural amino acids (i.e., compounds that do not occur in nature but that can be incorporated into a polypeptide chain; see, for example, http://www.cco.caltech.edu/^{˜}dadgrp/Unnatstruct.gif, which displays structures of non-natural amino acids that have been successfully incorporated into functional ion channels) and/or amino acid analogs as are known in the art may alternatively be employed). In certain embodiments, one or more of the amino acids in a protein may be modified, for example, by the addition of a chemical entity such as a carbohydrate group, a phosphate group, a farnesyl group, an isofarnesyl group, a fatty acid group, a linker for conjugation, functionalization, or other modification, etc.

“Protein:” As used herein, the term “protein” refers to a polypeptide (i.e., a string of at least two amino acids linked to one another by peptide bonds). Proteins may include moieties other than amino acids (e.g., may be glycoproteins, proteoglycans, etc.) and/or may be otherwise processed or modified. Those of ordinary skill in the art will appreciate that a “protein” can be a complete polypeptide chain as produced by a cell (with or without a signal sequence), or can be a characteristic portion thereof. Those of ordinary skill will appreciate that a protein can sometimes include more than one polypeptide chain, for example linked by one or more disulfide bonds or associated by other means. Polypeptides may contain L-amino acids, D-amino acids, or both and may contain any of a variety of amino acid modifications or analogs known in the art. Useful modifications include, e.g., terminal acetylation, amidation, methylation, etc. In some embodiments, proteins may comprise natural amino acids, non-natural amino acids, synthetic amino acids, and combinations thereof. The term “peptide” is generally used to refer to a polypeptide having a length of less than about 100 amino acids, less than about 50 amino acids, less than 20 amino acids, or less than 10 amino acids. In some embodiments, proteins are antibodies, antibody fragments, biologically active portions thereof, and/or characteristic portions thereof.

“Sample:” As used herein, the term “sample” typically refers to a biological sample obtained or derived from a source of interest, as described herein. In some embodiments, a source of interest comprises an organism, such as an animal or human. In some embodiments, a biological sample is or comprises biological tissue or fluid. In some embodiments, a biological sample may be or comprise bone marrow; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens; feces, other body fluids, secretions, and/or excretions; and/or cells therefrom, etc. In some embodiments, a biological sample is or comprises cells obtained from an individual. In some embodiments, obtained cells are or include cells from an individual from whom the sample is obtained. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, feces etc.), etc. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to techniques such as amplification or reverse transcription of mRNA, isolation and/or purification of certain components, etc. In certain embodiments, the sample is a uterine washing.

“Substantially”: As used herein, the term “substantially”, and grammatic equivalents, refer to the qualitative condition of exhibiting total or near-total extent or degree of a characteristic or property of interest. One of ordinary skill in the art will understand that biological and chemical phenomena rarely, if ever, go to completion and/or proceed to completeness or achieve or avoid an absolute result.

“Subject”: As used herein, the term “subject” includes humans and mammals (e.g., mice, rats, pigs, cats, dogs, and horses). In many embodiments, subjects are mammals, particularly primates, especially humans. In some embodiments, subjects are livestock such as cattle, sheep, goats, cows, swine, and the like; poultry such as chickens, ducks, geese, turkeys, and the like; and domesticated animals particularly pets such as dogs and cats. In some embodiments (e.g., particularly in research contexts) subject mammals will be, for example, rodents (e.g., mice, rats, hamsters), rabbits, primates, or swine such as inbred pigs and the like.

Drawings are presented herein for illustration purposes, not for limitation.

### BRIEF DESCRIPTION OF DRAWINGS

FIG. 1 is a Single Walled Carbon Nanotube (SWCT) antibody attachment scheme, according to an illustrative embodiment of the invention. Attachment of antibody to the biosensor has been achieved using DNA in order to both non-covalently encapsulate the carbon nanotube and to covalently attach the antibody. Photoluminescence plots (PL) show the fluorescence emission of multiple DNA-encapsulated nanotube species, before and after antibody conjugation.

FIG. 2 is a biomarker binding scheme (left), according to an illustrative embodiment of the invention. (Right) Detection of uPA antigen by the biosensor complex (solid line) if achieved by both ratiometric changes in the nanotube fluorescence intensity, as well as red-shifting of the fluorescence wavelength (inset). 100 nM uPA or BSA as a negative control was added; the measurement was taken 30 minutes after introducing the nanotube to the sensor.

FIG. 3 shows (top) specific detection by fluorescence emission blue shift of HE4 with antibody-functionalized SWCNT. FIG. 3 (bottom) shows a necessity of antibody functionalization for specific HE4 biomarker detection. SWCNT with no antibody were incubated with 100 nM BSA or HE4 and showed a non-specific red shift, while comparison with HE4 binding to antibody-functionalized SWCNT shows specific blue shift.

FIG. 4 shows BSA-coated anti-uPA nanotubes with 20× BSA coating for 30 minutes. Wavelength shift occurred upon addition of increasing uPA concentrations up to 100 nM.

FIG. 5 shows wavelength red shift of nanotube fluorescence emission as a function of uPA added to FBS (mean ±SD).

FIG. 6 shows wavelength shift of nanotube fluorescence emission as a function of HE4 added to FBS (mean±SD).

FIG. 7 shows sensor response in 10% human serum/HEP plasma/EDTA plasma with 100 nm uPA spiked. Y axis is change from non-spiked control. Data shows optimization of human blood sample type for sensor functionality. Data shown after 30 minutes.

FIG. 8 shows sensor response (1275 nm peak) in 10% human whole blood with 100 nm uPA (or control) spiked into sample. Measurement was taken after 90 minutes. Specific detection of 100 nM uPA in whole blood is shown.

FIG. 9 shows (left) a photoluminescence plot of fluorescence nanotubes in an alginate matrix. (Right) Fluorescence micrograph of nanotubes in al alginate matrix. Each bright focus is visualized fluorescence from nanotubes suspended in solution and embedded in the matrix (scale bar=10 μm).

FIG. 10 shows in vivo nanotube fluorescence hyperspectral imaging. (Left) Image of total nanotube fluorescence (900-1400 nm) in vivo. The region from which the nanotube spectrum was obtained is marked. (Right) Near-IR fluorescent spectrum from SWCT in vivo.

FIG. 11 is an exemplary design and function of an implantable biosensor for biomarker quantification within the inferior vena cava, according to an illustrative embodiment of the invention. (Left) The nanosensor can be immobilized on a device similar to clinically-available inferior vena cava filters. (Center) The immobilized device can be placed into the inferior vena cava accessed through the venous system via a catheter. Nanotube fluorescence response can be monitored from outside the body for quantitative changes due to biomarker binding. (Right) A quantitative response similar to as depicted here can be obtained from the nanotube fluorescence a biomarker concentration can be given.

FIG. 12 shows in vivo detection of ovarian cancer biomarker HE4 in mice injected with 100 nM HE4 versus mice injected with 100 nM BSA as a control.

FIGS. 13A-13B show in vivo detection of the ovarian cancer biomarker HE4 in mice bearing HE4-expressing tumors (OvCar-3) versus those bearing HE4 non-expressing tumors (Skov3).

FIG. 13A shows detection of HE4 in live mice after implantation of the sensor encapsulated in an implantable membrane.

FIG. 13B shows confirmation of HE4 detection in the implanted membrane ex vivo.

FIG. 14 is a schematic of a system comprising an excitation light source, a device comprising a SWCNT sensor (in vivo or ex vivo), and a detector, according to an illustrative embodiment of the invention.

FIGS. 15A-15G show design and in vitro characterization of optical nanosensor for HE4, according to an illustrative embodiment of the invention.

FIG. 15A is a scheme of Ab-DNA-SWCNT complex synthesis and proposed nanosensor function, according to an illustrative embodiment of the invention.

FIG. 15B shows a correlogram from dynamic light scattering showing correlation coefficient of successive measurements as a function of time for pre- and post-Ab-conjugated ssDNA-SWCNT. A larger correlation coefficient is due to larger particle size. Three measurements for each complex are shown.

FIG. 15C shows an electrophoretic light scattering of ssDNA-SWCNT before and after anti-HE4 antibody conjugation. A more positive surface charge is seen with antibody conjugation, as expected. Each bar represents mean of three measurements±SD. **=p<0.01; t-test.

FIG. 15D shows absorbance spectra of the hybridized ssDNA-SWCNT before and after conjugation of the anti-HE4 antibody. Inset: Photoluminescence (PL) plot showing emission spectra at successive laser excitation lines of the Ab-DNA-SWCNT sensor, showing multiple bright nanotube species present.

FIG. 15E shows a dose-response curve of nanotube emission as a function of HE4 concentration in 10% FBS. Each point is the mean of three experiments±SD.

FIG. 15F shows a response of the Ab-DNA-SWCNT complex to interferent proteins. Each bar is the mean of three experiments±SD. Control and HE4 p=1.01E⁻⁵; Control and BSA p=0.999; Control and CA125 p=0.302; Control and uPA p=2.64E⁻³; Control and FBS p=0.343; two-sided one-way ANOVA with Tukey post-hoc analysis.

FIG. 15G shows a kinetic response of nanotube emission upon introducing recombinant HE4.

FIGS. 16A-16E show a single-sensor hyperspectral HE4 assay of patient biofluids.

FIG. 16A shows an image of adsorbed Ab-DNA-SWCNT complexes. Scale bar=5 μm.

FIG. 16B shows spectra of single complex, denoted in FIG. 16A, before and 10 mins after introducing recombinant HE4.

FIG. 16C shows a shift in sensor emission wavelength 10 mins after addition of recombinant HE4 or 10% FBS. Pre-FBS to post-FBS*=p=0.03; Pre-HE4 to post-HE4 *=p=0.04; two-sided t-test. N=82 single nanotubes before and 98 after FBS, 100 before and 97 after HE4; data shown is mean±SEM.

FIG. 16D shows a sensor response to serum from HGSC three patients or healthy donors. HE4 concentrations, measured independently via ELISA, are specified for each sample. *=p=0.015; two-sided t-test.

FIG. 16E shows a sensor response to serum from HGSC patients or benign pelvic fluid. HE4 concentrations, measured independently via ELISA, are specified for each sample, with one exception due to sample volume limitation*=p=0.03; two-sided t-test.

FIGS. 17A-17F show an implantable nanosensor device, according to an illustrative embodiment of the invention.

FIG. 17A shows a semipermeable 500 kDa MWCO membrane capillary incorporating Ab-DNA-SWCNT complexes. Spectrum of the nanosensor acquired through the capillary wall.

FIG. 17B shows emission wavelength from the sensor after introducing recombinant HE4. Data shown is mean±SD of three measurements. ***=p=7.2E⁻⁶; two-sided t-test.

FIG. 17C shows a near-infrared image of nanosensor emission from the implanted device, overlaid onto a reflected light image of the mouse.

FIG. 17D is a schematic of probe-based system used to excite/acquire NIR emission from the implanted sensor in mice, according to an illustrative embodiment of the invention. Insert shows a typical spectrum of nanosensor emission acquired from the mouse.

FIG. 17E shows a photograph of data acquisition from the probe.

FIG. 17F shows a change in emission wavelength acquired from mice following i.p. injection of either 10 pmole BSA or HE4, compared to uninjected mice. Each bar is the mean of measurements from three mice±SD. *=p=0.016; two-sided t-test.

FIGS. 18A-18C show in vivo detection of HE4 in ovarian cancer models.

FIG. 18A shows bioluminescence images denoting tumor burden in the peritoneal cavity of nude mice inoculated with luciferase-expressing cell lines.

FIG. 18B shows representative ELISA results of HE4 concentration in ascites collected from mice peritoneal cavities.

FIG. 18C shows sensor response from all mice. Bars represent the mean of four mice used for each condition in the experiment±SD. Sensor wavelength difference between models and statistical analysis: SK-OV-3 and OVCAR-3 (0.64 nm; p=0.020); SK-OV-3 and OVCAR-5 (1.1 nm; p=3.8E⁴); OVCAR-8 and OVCAR-3 (0.79 nm; p=0.0048); OVCAR-8 and OVCAR-5 (1.2 nm; p=1.2E⁴); two-sided one-way ANOVA with Tukey post-hoc analysis.

FIGS. 19A-19D show characterization of sensor function in vitro.

FIGS. 19A and 19B each is a dose-response curve of nanotube emission as a function of HE4 concentration in 10% FBS for (FIG. 19A) (8,6) and (FIG. 19B) (8,7) nanotube species. Each point is the mean of three experiments±SD.

FIG. 19C shows center wavelength time-course measurements every 5 minutes correlating to the experiment shown in FIG. 15E reveal stabilization of sensor blue-shift at approximately 60 minutes and stable sensor function when other proteins are added.

FIG. 19D shows a center wavelength of the DNA-SWCNT complex in 10% FBS with no additional protein added or with 500 nM HE4 added. Data reveals no change in nanotube center wavelength upon addition of HE4 when the specific anti-HE4 antibody is not present. Each bar represents the mean of three measurements±SD.

FIG. 20 shows a Lorentzian fit of the sum of binned center wavelengths for individual nanotubes before and after addition of HE4 to the immobilized sensors.

FIG. 21 shows images of the procedure to surgically implant the sensor devices.

FIG. 22 shows a concentration of HE4 in conditioned cell culture media as determined by ELISA for SK-OV-3, OVCAR-8, OVCAR-3, and OVCAR-5 cells.

FIG. 23 shows a haemotoxylin and eosin (H&E) stain of tumor nodules from each in vivo model of ovarian cancer. All scale bars represent 100 μm.

### DETAILED DESCRIPTION

Described herein are compositions useful for the detection of analytes. In certain embodiments, the invention relates to DNA-encapsulated single-walled carbon nanotubes (SWCNTs) functionalized with an antibody or other analyte-binding species, for detection and/or imaging of an analyte in a biological sample or subject. Other embodiments described herein include systems, methods, and devices utilizing such compositions for ex vivo biomarker quantification, tissue optical probes, and in vivo analyte detection and quantification.

### Sensors

Described herein are compositions, systems, devices, and methods comprising a single-walled carbon nanotube (SWCNT) sensor.

### Single-Walled Carbon Nanotubes (SWCNTs)

Described herein are devices and methods comprising single-walled carbon nanotubes (SWCNTs). SWCNTs are rolled sheets of graphene with nanometer-sized diameters. SWCNTs are defined by their chirality. The sheets that make up the SWCNTs are rolled at specific and discrete, i.e., “chiral” angles. This rolling angle in combination with the nanotube radius determines the nanotube's properties. SWCNTs of different chiralities have different electronical properties. These electronic properties are correlated with respective differences in optical properties. Thus, individually-dispersed semiconducting SWCNTs exhibit ideal qualities as optical biomedical sensors.

Semiconducting SWCNTs are fluorescent in the near-infrared (NIR, 900-1600 nm) due to their electronic band-gap between valence and conduction band. The semiconducting forms of SWCNTs, when dispersed by surfactants in aqueous solution, can display distinctive near-infrared (IR) photoluminescence arising from their electronic band gap. IR is a wavelength range penetrant to tissue, and thus potentially suitable for implantable sensors or other devices. The band-gap energy is sensitive to the local dielectric environment around the SWCNT, and this property can be exploited in chemical sensing.

### Nucleotides

In certain embodiments, the sensor as described herein comprises a polymer capable of being non-covalently or covalently conjugated to the SWCNT. In certain embodiments, the polymer is DNA, RNA, an artificial nucleic acid including peptide nucleic acid (PNA), Morpholino, locked nucleic acid (LNA), glycol nucleic acid (GNA), threose nucleic acid (TNA), an amino-acid sequence, or a synthetic monomer

In certain embodiments, the sensor as described herein comprises a nucleotide attached to the SWCNT. In certain embodiments, the nucleotide can have fewer than 100,000, fewer than 50,000, fewer than 25,000, fewer than 10,000, fewer than 5,000, fewer than 1,000, fewer than 500, fewer than 250, fewer than 100, fewer than 75, fewer than 50, fewer than 30, fewer than 25, fewer than 20, 15, 12, 10, 8, 6 or 4 nucleotides.

In certain embodiments, the nucleotide can have a random sequence. In certain embodiments, the nucleotide can have an ordered sequence. In certain embodiments, the ordered sequence can be a predetermined sequence. In certain embodiments, the ordered sequence can be a repeating sequence. In certain embodiments, the repeat sequence can include fewer than 500, fewer than 400, fewer than 300, fewer than 200, fewer than 100, fewer than 50, fewer than 30, fewer than 25, fewer than 20, 15, 14, 13, 12, 11, 10, 9, 8, 7, 6, 5, 4, 3 or 2 nucleotides. In certain embodiments, the polynucleotide can be poly(AT), poly(GT), poly(CT), poly(AG), poly(CG), or poly(AC). In certain embodiments, the polynucleotide can have a content. In certain embodiments, the content can be a percentage of a unique nucleotide present in the sequence. In certain embodiments, the nucleotide sequence is a single-stranded DNA molecule (ssDNA).

### Targets and Analytes

Target conditions and diseases that can be diagnosed or otherwise assessed using the devices and methods described herein include, for example, cancers (including tumors), metabolic disease, fetal health condition, kidney disease, organ rejection, hereditary diseases, nervous disease, obesity, and infectious disease. In certain embodiments, the condition or disease is at least in part characterized by a substance, i.e., an analyte.

In certain embodiments, the analytes that can be detected, imaged, mapped, or quantified using the systems, devices, and methods described herein include peptides, polypeptides, proteins, biologics, biomolecules, biosimilars, aptamers, viruses, drugs, lipids, bacteria, toxins, cells, tumor cells, cancer, antibodies, and antibody fragments.

In certain embodiments, the analytes are biomarkers for ovarian cancer (e.g., HE4, e.g., CA-125, e.g., mesothelin, e.g., CRABP2 (cellular retinoic acid binding protein 2), e.g., YKL-4, e.g., and any combinations thereof). In certain embodiments, the analytes are biomarkers for prostate cancer and/or other metastatic cancer states (e.g., uPAR (uPA receptor), e.g., uPA (urokinase plasminogen activator)). In certain embodiments, the analytes are biomarkers for ovarian cancer, breast cancer, and/or lung cancer (e.g., CA125). In certain embodiments, the analytes are biomarkers for ovarian cancer, pancreatic cancer, and/or breast cancer (e.g., HE4). In certain embodiments, the analytes are biomarkers for ovarian cancer and/or other diseases (e.g., mesothelin, e.g., YKL-40). In certain embodiments, the analytes are biomarkers for prostate cancer (e.g., PSA (prostate specific antigen). In certain embodiments, the analytes are biomarkers for prostate cancer and/or other cancers (e.g., PSMA (prostate specific membrane antigen)). In certain embodiments, the analytes are biomarkers for a variety of cancers such as pancreatic cancer, breast cancer, and/or lung cancer (e.g., carcinoembryonic antigen (CEA)). In certain embodiments, the analytes are biomarkers for breast cancer and/or other cancers (e.g., MUC1).

### Systems, Devices, and Methods

In certain embodiments, the device is a sensing platform. In certain embodiment, the device is a sensor. In certain embodiments, the device is in contact with a biofluid or bodily fluid sample. In certain embodiments, the bodily fluid sample is e.g., Amniotic fluid, Aqueous humour and vitreous humour, Bile, Blood serum, Breast milk, Cerebrospinal fluid, Cerumen (earwax), Chyle, Chyme, Endolymph and perilymph, Exudates, Feces, Female ejaculate, Gastric acid, Gastric juice, Lymph, Menstrual fluid, Mucus (including nasal drainage and phlegm), Pericardial fluid, Peritoneal fluid, Pleural fluid, Pus, Rheum, Saliva, Sebum (skin oil), Serous fluid, Semen, Smegma, Sputum, Synovial fluid, Sweat, Tears, Urine, Vaginal secretion, Vomit., etc. In certain embodiments, the bodily fluid in contact with the device is not treated or purified prior to contact with the device.

In certain embodiments, the device is a sensor, or comprises a sensor, as described herein, wherein the device is placed outside of an organism to be treated or diagnosed. In certain embodiments, the device is a point-of-care diagnostic device, a wearable device, or a piece of laboratory equipment. In certain embodiments, the device can be positioned on the surface of the organism, such as the arm, and, e.g., worn like a wristwatch. In certain embodiments, the device is implantable into the organism. In certain embodiments, the devices is a point-of-care medical device, e.g., a (urine) dipstick, a test strip, a membrane, a skin patch, a skin probe, a gastric band, a stent, a catheter, a needle, a contact lens, a prosthetic, a denture, a vaginal ring, or other implant. In certain embodiments, the device comprises a solid support, a membrane, a gel, or a microfluidic component. In certain embodiments, the device comprises a microfluidic chamber containing a sensor. In certain embodiments, the device comprises a sensor contained in a semi-permeable enclosure.

In certain embodiments, the organism to be treated or diagnosed is a mammal, a human, a dog, a rodent, or a farm animal. In certain embodiments, the device is used in to detect oligonucleotides in vivo with a noninvasive method. In certain embodiments, the method is a real-time, non-invasive monitoring in vivo.

In certain embodiments, the device is a sensor, or comprises a sensor, as described herein, and is exposed excitation electromagnetic radiation (excitation EMR) to produce an emission of electromagnetic radiation (emission EMR) by the SWCNT sensor. In certain embodiments, the excitation EMR is ultraviolet light, infrared light, or near-infrared light (NIR). In certain embodiments, the excitation EMR is visible light. In certain embodiments, the excitation EMR has a wavelength between 100 nm and 3000 nm, 200 nm and 2000 nm, between 300 and 1500 nm, or between 500 and 1000 nm.

In certain embodiments, the emission EMR is ultraviolet light, infrared light, or near-infrared light (NIR). In certain embodiments, the emission EMR is visible light. In certain embodiments, the emission EMR has a wavelength between 300 nm and 3000 nm, between 400 and 2000 nm, between 500 and 1500 nm, between 600 nm and 1400 nm, or between 700 and 1350 nm.

In certain embodiments, the methods described herein can be used for diagnostic or therapeutic purposes to diagnose, prevent, or treat any condition or disease characterized by or associated with an analyte as described herein. In certain embodiments, the method comprises contacting a test sample comprising one or more analytes of interest; exposing the test sample to excitation electromagnetic radiation (excitation EMR) to produce an emission of electromagnetic radiation (emission EMR) by the SWCNT sensor; detecting the electromagnetic radiation emitted by the SWCNT sensor; and identifying the presence of the one or more analytes of interest in the test sample based at least in part on the detected emission EMR. Sources of excitation EMR can be any such source known in the art, e.g., a laser, a light emitting diode, or a lamp. Detectors of emission EMR can be any such detector known in the art, e.g., a fluorometer. In certain embodiments, the method comprises detecting a wavelength shift (e.g., a blue or red shift) in the emission EMR and/or an intensity shift (e.g., amplitude shift), or other changes in the spectral characteristics of in the emission EMR, thereby identifying the presence of the species having the target nucleotide sequence in the test sample.

In certain embodiments, the method comprises detecting an intensity shift between an emission center wavelength (e.g., a peak) of the test sample and an emission center wavelength (e.g., a peak) of a reference sample, wherein the reference sample is devoid of the species having the target nucleotide sequence. In certain embodiments, the emission wavelength shift is between 1 nm and 100 nm, between 2 nm and 100 nm, between 3 and 50 nm, or between 4 and 20 nm. In certain embodiments, the wavelength shift is a color shift, e.g., a redshift or a blueshift. In certain embodiments, the wavelength shift is a blueshift.

In certain embodiments, the device is a sensor, or comprises a sensor, as described herein, and is a device for a non-medical application. In certain embodiments, the device is a device for monitoring environmental conditions. In certain embodiments, the device comprises a solid support, a membrane, a gel, or a microfluidic component, or a combination thereof In certain embodiments, the device comprises a microfluidic chamber containing a sensor. In certain embodiments, the device comprises a sensor contained in a semi-permeable enclosure.

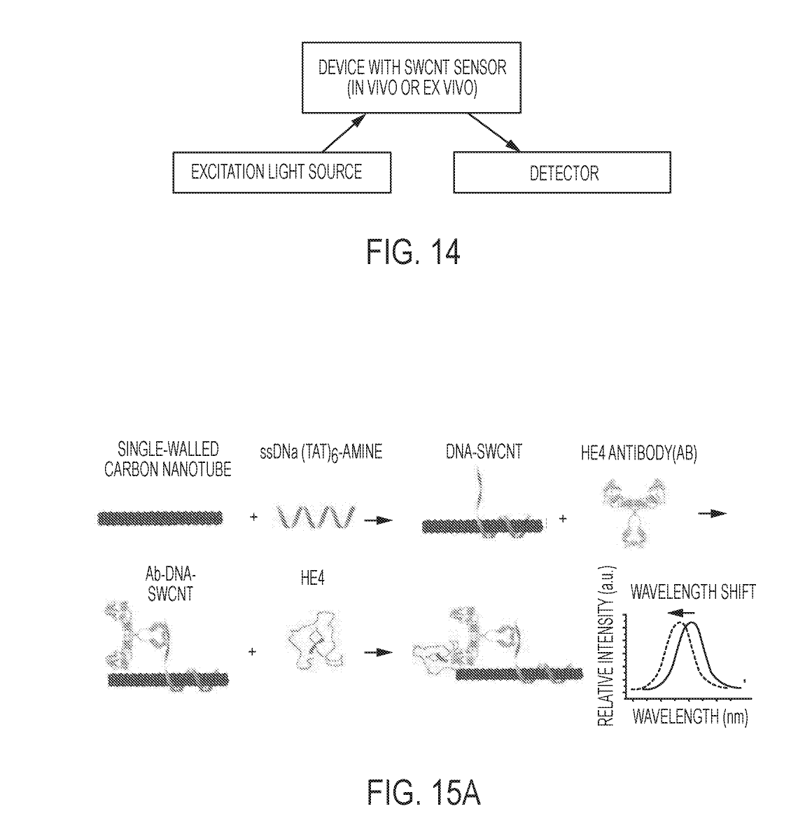

FIG. 14 is a schematic depicting a system with an excitation light source, a device with a SWCNT sensor as described herein, and a detector, according to an illustrative embodiment.

### EXPERIMENTAL EXAMPLES

### Carbon Nanotube-Based Antibody Sensors Sensitive and Specific for Cancer Biomarkers

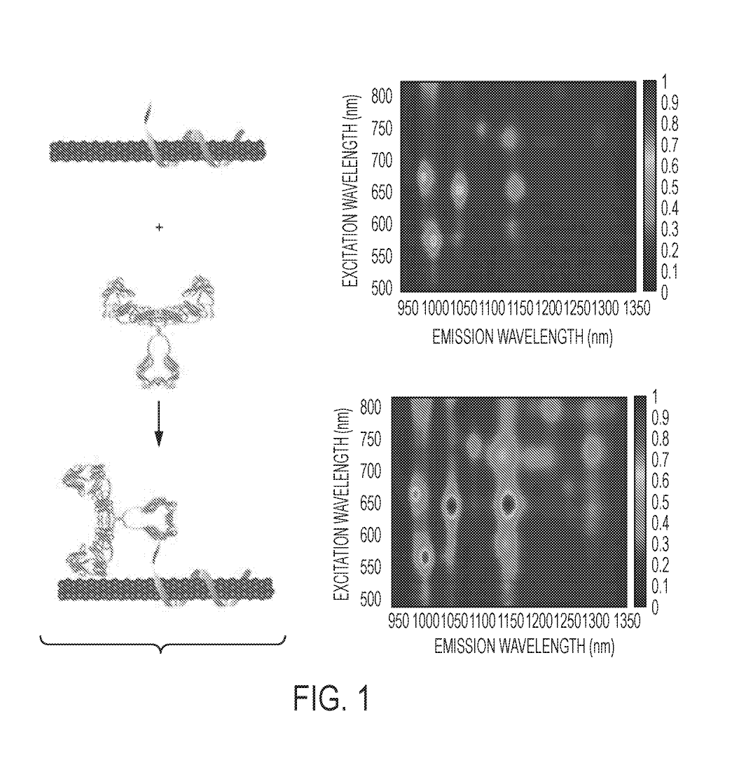

Experiments have been conducted with SWCNT biosensors for uPA (urokinase plasminogen activator) and HE4 (human epididymis protein 4), characterized in vitro. SWCNTs were suspended in solution with ssDNA oligonucleotides. Amine-modified DNA oligomers, of predetermined sequence and quantity, were commercially synthesized and purchased (IDT DNA). In the HiPCO SWCNT sample (Nanolntegris), there exists more than 12 chiralities of semi-conductive nature. This complex was functionalized with a commercially-available (Santa Cruz Biotechnologies) anti-uPA antibody or anti-HE4 antibody (RayBiotech) by EDC/NHS activation of the carboxylic acid groups on the antibody. These activated groups were conjugated to amine-functionalized DNA encapsulating SWCNT via a simple amidation reaction. Unconjugated antibody was dialyzed away to obtain purified antibody-DNA-SWCNT complexes (FIG. 1). Verification of conjugation was performed by fluorescent and absorbent spectroscopy as well as dynamic light scattering.

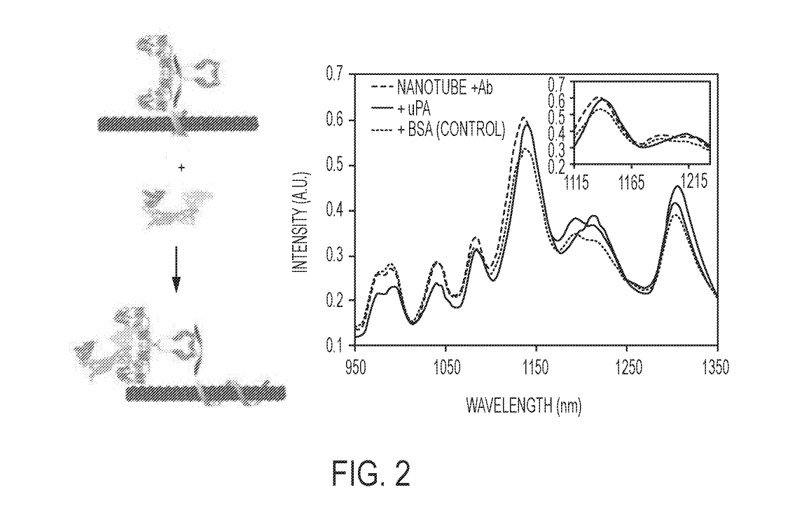

To initially test the sensitivity and specificity of the sensor complexes, they were challenged with increasing concentrations of uPA or HE4 in phosphate-buffered saline (PBS). 0.1-100 nM commercially-obtained recombinant uPA (RayBiotech) was added to the sensor complex, and nanotube fluorescence was monitored with laser excitation 500-800 nm and emission 900-1400 nm. Additionally, the uPA sensor complex was challenged with bovine serum albumin (BSA) as a control for non-specific binding (FIG. 2).

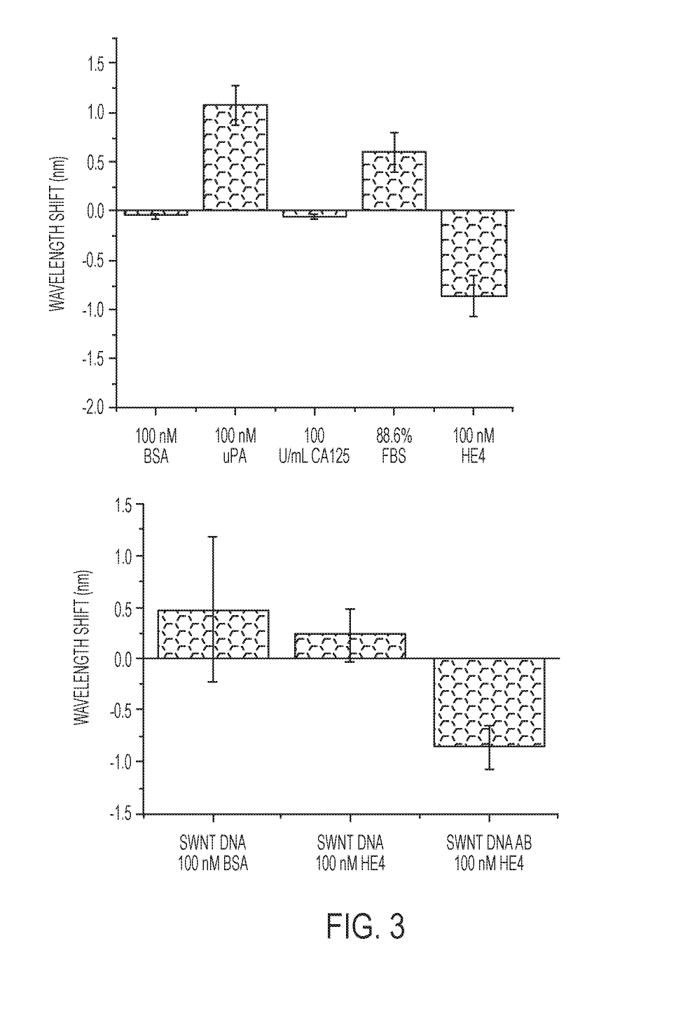

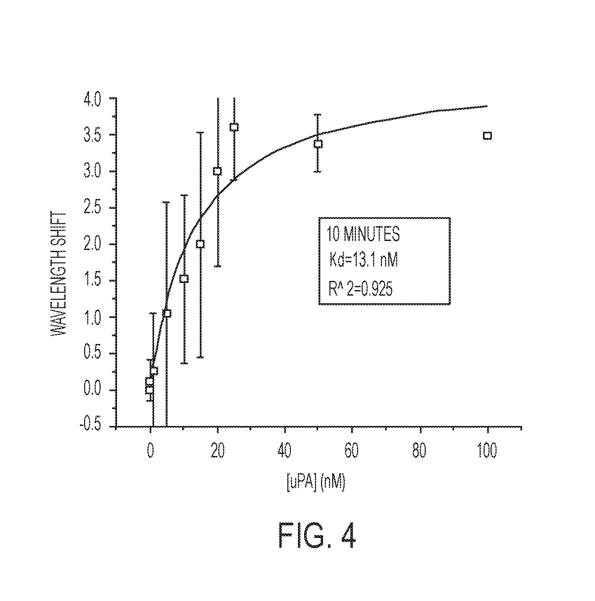

The HE4 sensor complex was also challenged with 100 nM BSA, 100 nM uPA, 100 U/mL CA125, and 88.6% fetal bovine serum to show specificity of the sensor (FIG. 3). To determine that specificity is conferred by the antibody conjugation, SWCNT encapsulated with DNA having no conjugated antibodies was used to show that 100 nM BSA and 100 nM HE4 do not cause the specific binding signal as seen with the antibody-conjugated sensor (FIG. 3). For the uPA sensor, it was observed that the biosensor exhibited a specific wavelength redshift in individual nanotube fluorescence emission when spiked with uPA. From this response, a dose response curve was obtained with a dissociation constant (Kd) of 13.1 nM and a dynamic range of 5-50 nM, which encompasses serum uPA levels for metastatic detection (FIG. 4).

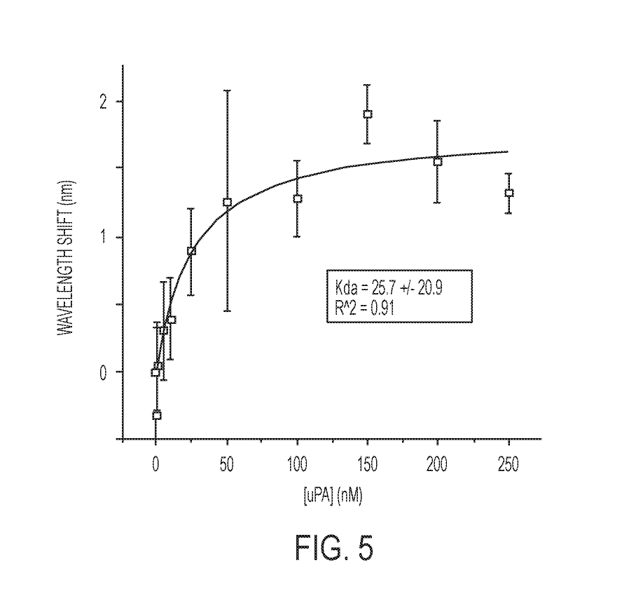

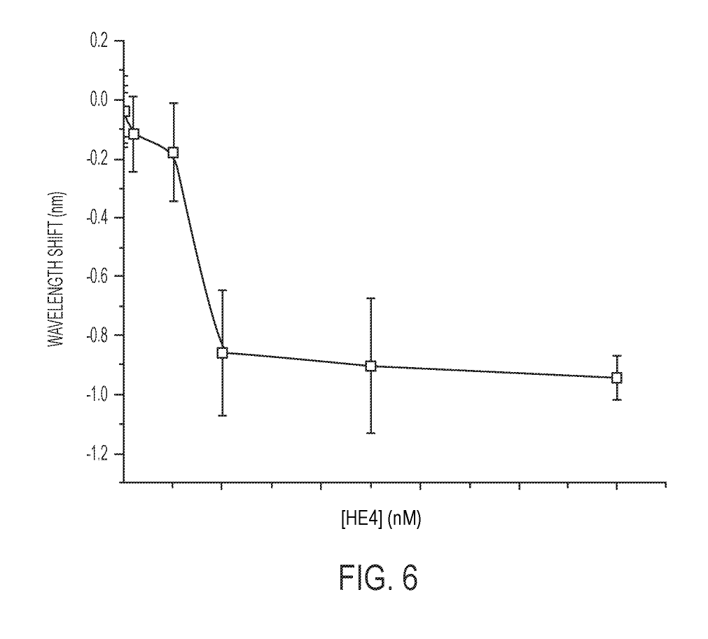

The sensor complexes were also challenged with uPA or HE4 in 10% fetal bovine serum (FBS), used here as an analogue of the complex matrix of human blood. The sensing properties of SWCNTs are not harmed by media which is strongly scattering and/or absorbing and are thus ideal for us in complex matrices. The FBS solution was spiked with a clinically-relevant concentration range of specific biomarker. Fluorescent excitation and emission was performed as above to analyze the response of individual SWCNT species. The uPA sensor exhibited the same wavelength redshift when spiked with uPA, from which a dose response curve with a Kd of 24.1 nM and an identical dynamic range of 5-50 nM was obtained (FIG. 5). The HE4 sensor showed a monotonic blue shift in SWCNT fluorescence wavelength upon addition of increasing HE4 concentrations in a clinically-relevant dynamic range of 10-100 nM (FIG. 6). Thus, each sensor is operational in the ideal conditions of PBS as well as in FBS in a range relevant to clinical biomarker detection.

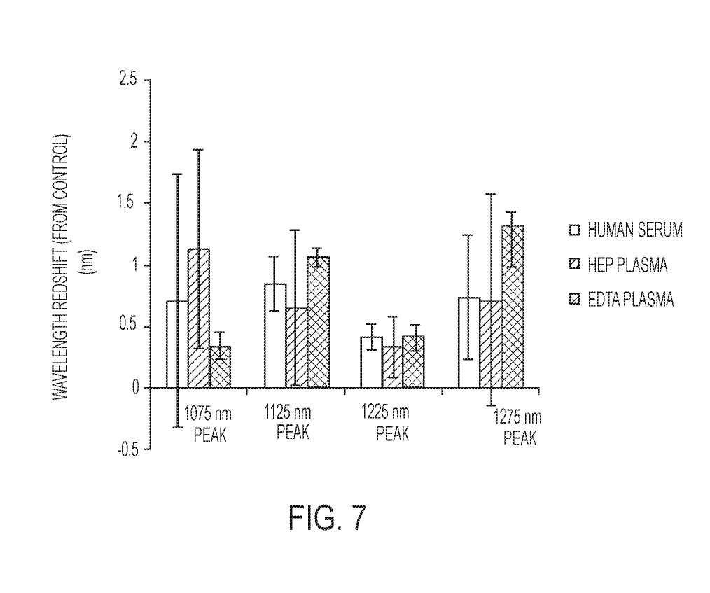

Experiments were also conducted to test uPA sensor response in 10% human serum, heparinized plasma, and EDTA plasma (FIG. 7). Each sample was spiked with 100 nM uPA, and the wavelength redshift from a non-spiked matched sample was obtained. It was possible to specifically detect a 100 nM uPA difference in clinical samples. Furthermore, the sensor response is most optimal in EDTA plasma, with a 1.5 nm redshift of the 1275 nm nanotube peak after 30 minutes of incubation. The heparinized plasma sample showed greater variation and thus less specific and robust response. This was expected as heparin has been demonstrated to tightly bind uPA.

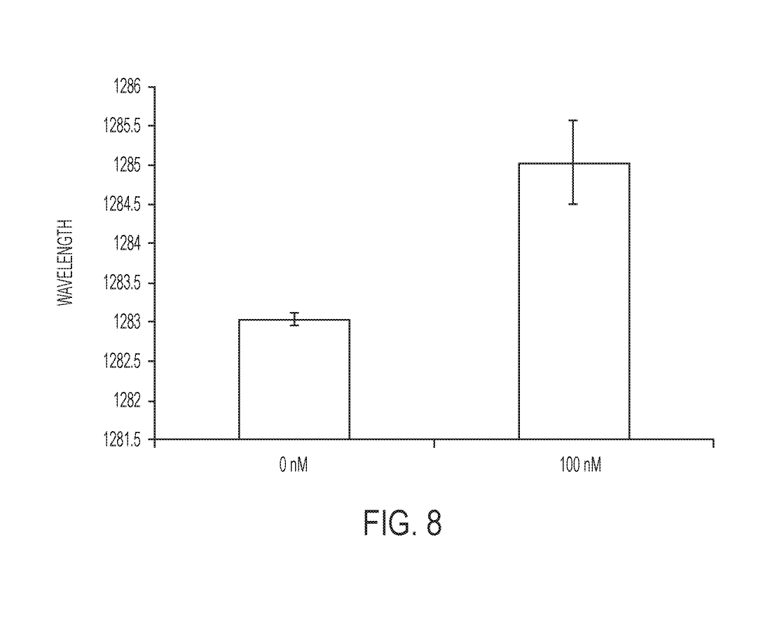

The sensor also displayed a significant response of approximately 2 nm redshift upon addition of 100 nM uPA to 10% whole human blood compared to non-spiked control (FIG. 8). This result is particularly significant as only limited work has been performed to develop whole-blood biomarker nanosensors due to complexity of the blood components.

### Carbon Nanotube-Based Sensors Can Be Immobilized and Imaged In Vivo

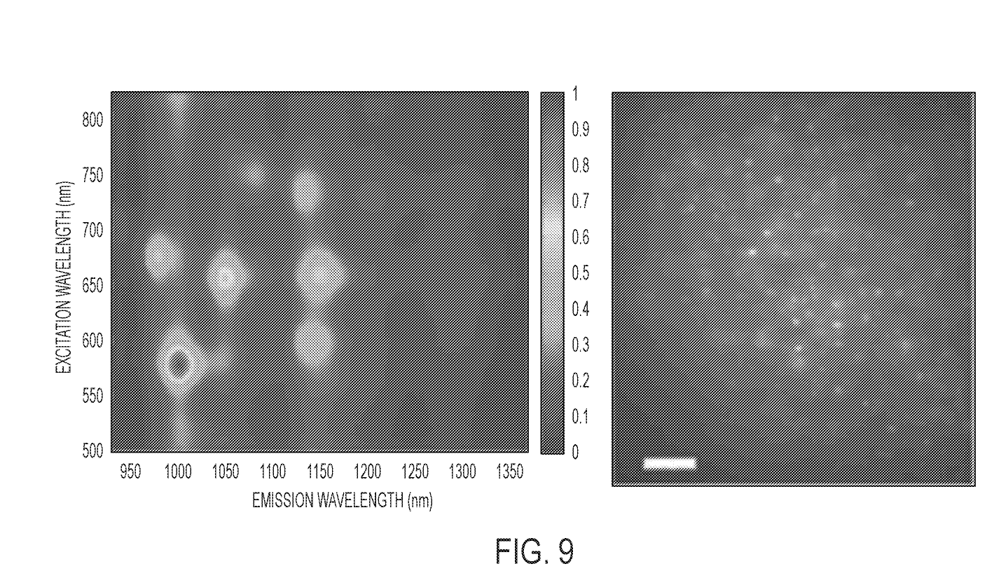

To date, ssDNA-encapsulated SWCNT have been immobilized in an alginate hydrogel and the NIR fluorescence emission characterized (FIG. 9). Commercially-available alginate was mixed with sodium bicarbonate in the presence of SWCNT and added dropwise into a 0.1 M calcium chloride solution in 10% acetic acid. The immobilized nanotube solution was interrogated for NIR fluorescence spectroscopically and microscopically. Thus, it is confirmed that carbon nanotubes can be immobilized and fluorescently characterized in an alginate matrix.

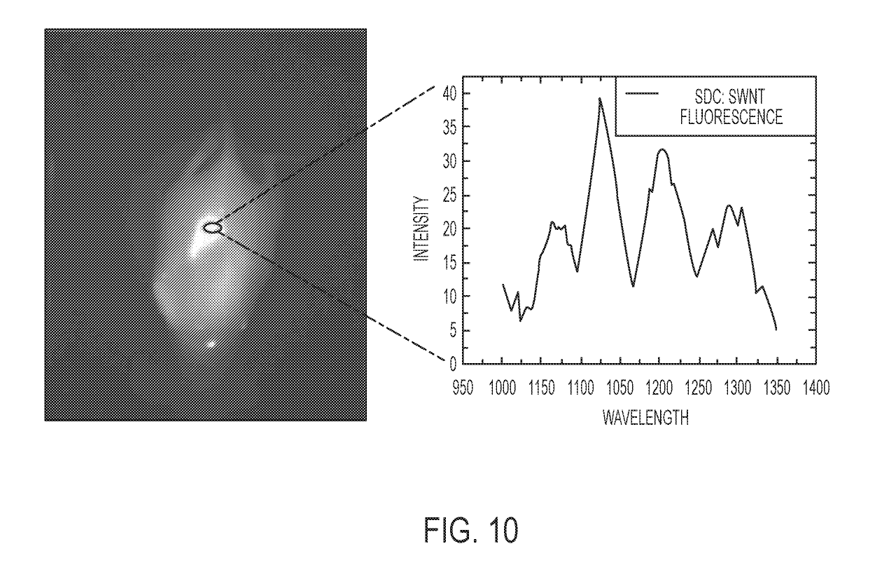

Experiments were conducted injecting free nanotube solution into SKH1-Elite hairless, immune competent mice. The mice were imaged with a custom pre-clinical NIR whole animal imaging system (Photon Etc.). With this system, a full NIR spectrum was obtained from multiple nanotube species in vivo (FIG. 10). Additionally, it is found that nanotube fluorescence is detectable through the entire width of the mouse. Importantly, it was found that the subcutaneously-injected nanotube solution did not cause any obvious toxic effects in mice over the course of at least three months, in agreement with previous work showing the relative lack of toxicity of SWCNT.

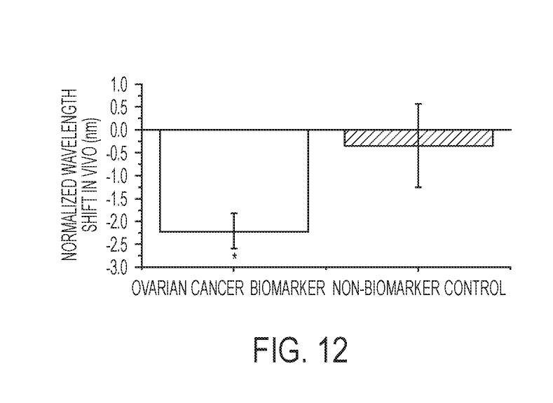

FIG. 12 shows results of in vivo detection of the ovarian cancer biomarker HE4 in mice injected with 100 nM HE4 versus mice injected with 100 nM bovine serum albumin as a control.

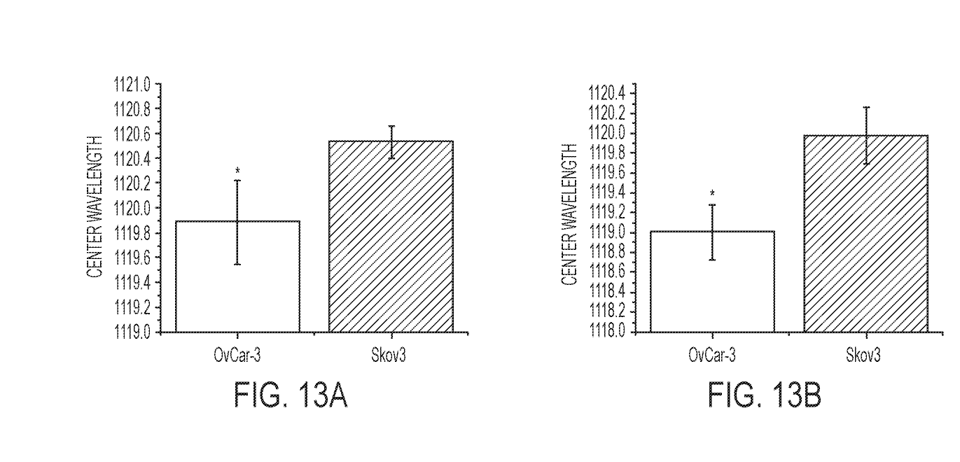

FIGS. 13-13B show results of in vivo detection of the ovarian cancer biomarker HE4 in mice bearing HE4-expressing tumors (OvCar-3) versus those bearing HE4 non-expressing tumors (Skov3). FIG. 13A shows detection of HE4 in live mice after implantation of the sensor encapsulated in an implantable membrane. FIG. 13B shows confirmation of HE4 detection in the implanted membrane ex vivo.

### Constructive Example: Human Sensor Immobilization and Biomarker Measurement

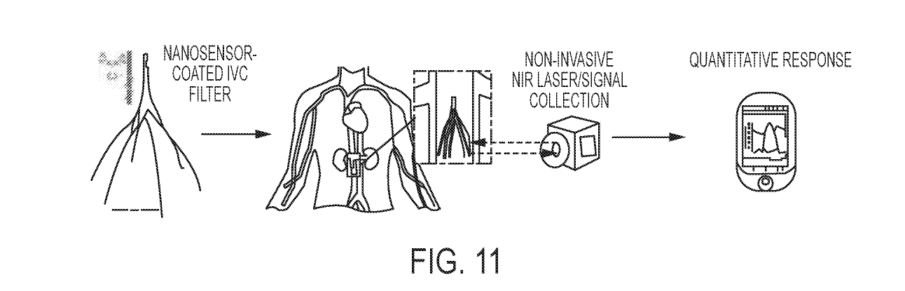

In certain embodiments, the developed biosensor can be immobilized on a device that is similar to inferior vena cava filters already in use in the clinic, or in another similar implantable device (FIG. 11). This device can be implanted through the femoral vein, internal jugular vein, or access points when compressed into a thin catheter. Sensor fluorescence signal can be monitored externally via a non-invasive near-infrared laser and signal collector. The fluorescence signal can be benchmarked via a standard calibration curve similar to that obtained in FIGS. 3 and 4 to obtain quantitative circulating biomarker concentrations. This would yield a measurement of the concentration of multiple biomarkers, allowing immediate, informed decisions to be made by the physician regarding patient treatment.

### EXAMPLE 1

### Detection of HE4 Using Carbon Nanotube-Based Antibody Sensors

Early-stage detection of high-grade serous ovarian cancer (HGSC) remains elusive, potentially because FDA-approved serum biomarkers CA125 and HE4 do not appear at detectable levels until advanced stages of the disease.

Without wishing to be bound to any theory, an implantable device placed proximal to disease sites, such as the fallopian tube, ovary, uterine cavity, or peritoneal cavity, may constitute a feasible strategy to improve detection of HGSC. A prototype optical sensor composed of an antibody-functionalized carbon nanotube complex which responds quantitatively to HE4 via modulation of the nanotube optical bandgap was engineered. The complexes measured HE4 with nanomolar sensitivity to differentiate disease from healthy patient biofluids, and a semi-permeable sensor-loaded capillary, implanted surgically into four models of ovarian cancer, enabled the detection of HE4 optically within the live animals. In this Example, the first in vivo optical nanosensor capable of non-invasive quantification of a cancer biomarker in a model of disease is presented.

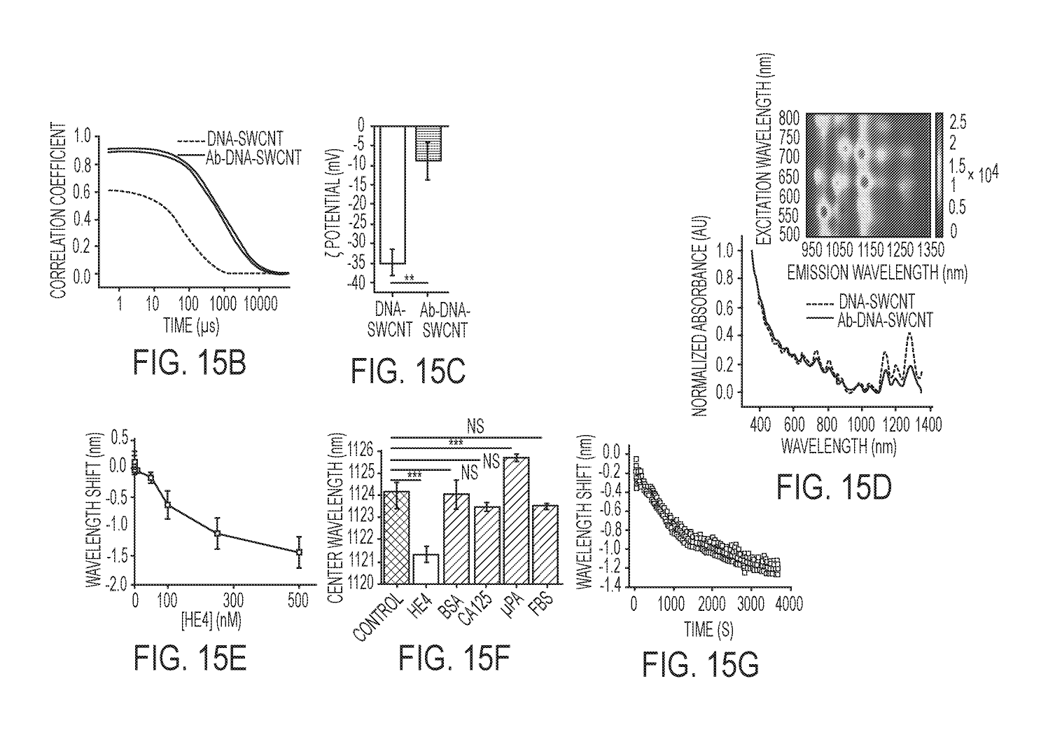

It was endeavored to develop a carbon nanotube-based sensor for HE4 by synthesizing a stable anti-HE4-nanotube complex without perturbation of the graphitic carbon of the nanotube (FIG. 15A). Single-walled carbon nanotubes (Unidym HiPCO preparation) were suspended with single-stranded DNA with the sequence (TAT)₆, modified at the 3′ end with an amine functional group via ultrasonication to form DNA-SWCNT suspensions. The nanotubes were purified by ultracentrifugation to remove bundles, and excess DNA was removed by centrifugal filtration. The DNA-SWCNTs were then conjugated via carbodiimide crosslinker chemistry to a goat polyclonal anti-HE4 IgG antibody (C-12, Santa Cruz Biotechnology) and subsequently dialyzed against water for 48 hours to remove unreacted reagents. Dynamic light scattering of the dialyzed suspensions before and after conjugation to the antibody showed that the complexes increased in size, confirming that the antibody was attached to DNA-SWCNT (FIG. 15B). Electrophoretic light scattering further supported successful attachment by an increase in -potential of the complex (FIG. 15C). The stability of the complexes and preservation of nanotube optical properties was confirmed by absorbance and photoluminescence excitation/emission spectroscopy (FIG. 15D). All nanotube species (chiralities) exhibited a red-shift in emission wavelength (red-shift) after antibody conjugation (Table 1), suggesting an increase in the local electrostatic charge and/or dielectric environment.

Table 1 shows a change in the nanotube emission wavelength of the DNA-SWCNT following conjugation of the anti-HE4 antibody to the DNA.

TABLE 1

| Chirality | Red-shift (nm) |
|---|---|
| 8, 3 | 0.86 |
| 6, 5 | 1.84 |
| 7, 5 | 1.98 |
| 10, 2 | 1.14 |
| 9, 4 | 2.36 |
| 8, 4 | 3.92 |
| 7, 6 | 1.92 |
| 8, 6 | 0.84 |

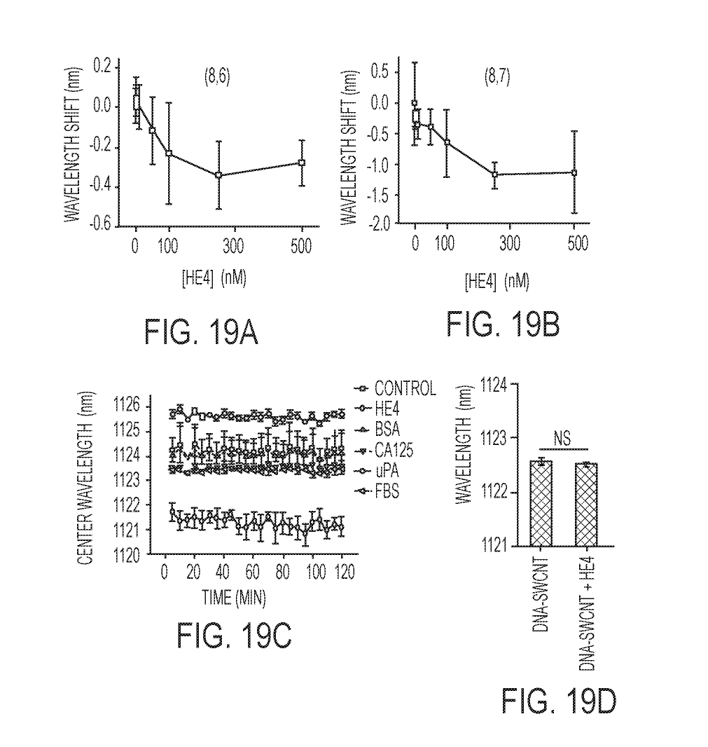

The sensitivity, specificity, and kinetics of the Ab-DNA-SWCNT complexes to HE4 were assessed. The complexes were passivated by incubating with bovine serum albumin (BSA) and interrogated with recombinant HE4 antigen in 10% fetal bovine serum (FBS) to approximate a complex protein environment. The complexes were excited at 730 nm, and the emission was collected across the NIR range of 900-1400 nm to assess several nanotube chiralities simultaneously (see Methods). The nanotube emission responded to increasing concentrations of HE4 via monotonic blue-shifting of the (9,4) nanotube chirality, and of the two other chiralities that were investigated, with a detection limit of 10 nM and sensitivity up to 500 nM (FIG. 15E; FIGS. 19A-19B). This detection limit is within the range found in ovarian cancer patient serum and ascites, which is up to 10 nM, and in uterine washings, which is up to 23-fold greater than maximal serum concentrations.

The specificity of the response of the Ab-DNA-SWCNT complex to HE4 was also investigated (FIG. 15F). The complex was interrogated with interferents including urokinase plasminogen activator (uPA), the ovarian cancer biomarker CA125, bovine serum albumin (BSA), and 93% fetal bovine serum (FBS), resulting in either no change or a moderate red-shifting response compared to the control (no protein). The responses were measured transiently, resulting in no further changes for 120 minutes (FIG. 19C). When ssDNA-suspended nanotubes in the absence of a conjugated antibody were challenged with HE4, no wavelength shifting response was observed, indicating that the ssDNA-suspended nanotubes did not exhibit an intrinsic response to HE4 (FIG. 19D).

The kinetics of the response of the Ab-DNA-SWCNT complexes to HE4 were assessed. The complexes exhibited an immediate change in wavelength after introducing HE4, which was detectable after 1 minute (FIG. 15G). The signal stabilized by approximately 60 minutes after HE4 addition.

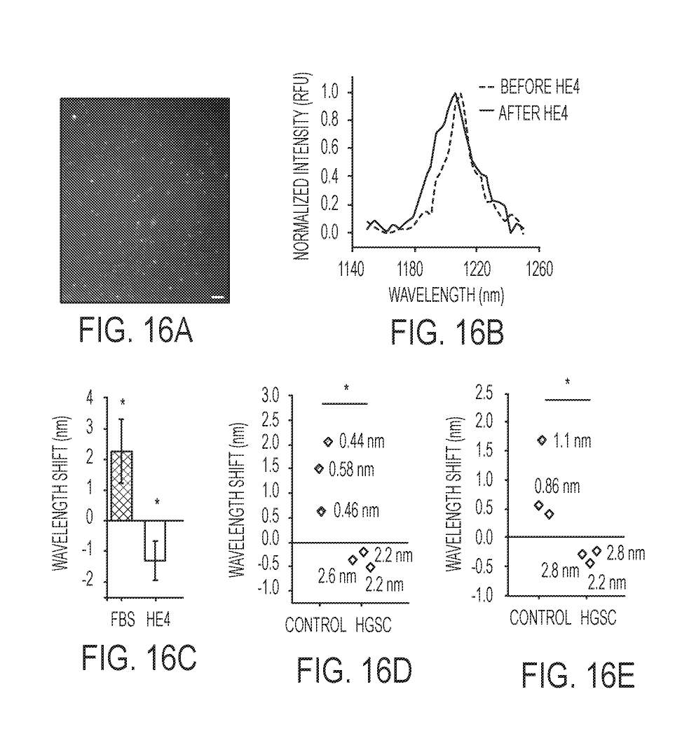

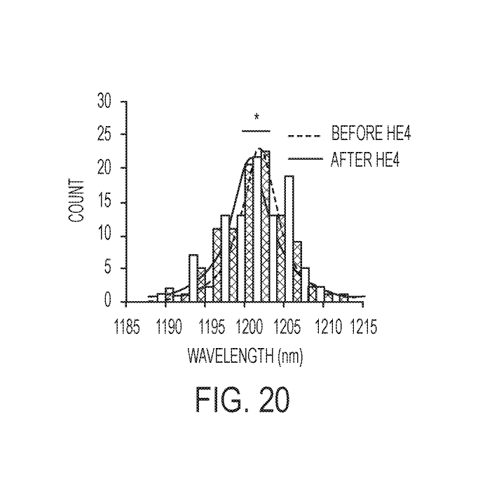

We developed a hyperspectral imaging-based assay to assess the response of single nanosensor complexes to HE4. Non-passivated Ab-DNA-SWCNT complexes were adsorbed to a glass surface and imaged by hyperspectral microscopy to rapidly acquire the spatially-resolved spectra from hundreds of individual complexes³⁴ (FIG. 16A). Baseline hyperspectral cubes were obtained from single nanotubes immersed in PBS, resulting in spectra for each complex. Spectra were then acquired from the same imaging field 10 minutes after spiking 10 μL of 100 nM HE4 into the buffer (FIG. 16C). The expected mean blue-shift was observed of 1.3 nm (p=0.04, measured for the (8,6) species) (FIG. 16C; FIG. 20). In response to 10% FBS, the mean sensor red-shift was 2.3 nm (p=0.03) (FIG. 16C).

The individual nanosensor response upon interrogation with biofluid samples collected from ovarian cancer patients was investigated. The spectral imaging assay was employed in part to minimize the volume of patient sample required. Upon interrogating the sensor with serum from HGSC patients and healthy donors, it was noticed that a distinct separation in signal response (FIG. 16D). The HGSC patient serum caused a blue-shift of approximately 0.36 nm (SD=0.16 nm, measured for the (8,6) nanotube species), while non-HGSC patient serum red-shifted the sensors by approximately 1.4 nm (SD=0.72 nm), resulting in a significant difference between the two cohorts (p=0.015). The nanosensor also differentiated between ascites collected from HGSC patients and peritoneal fluid collected from healthy control patients without a cancer diagnosis (FIG. 16E). Benign pelvic fluid from healthy controls resulted in an average red-shift of 0.96 nm (0.64 nm SD), while ascites from HGSC patients resulted in an average blue-shift of 0.27 nm (0.08 nm SD), resulting in a significant difference between the two populations (p=0.030).

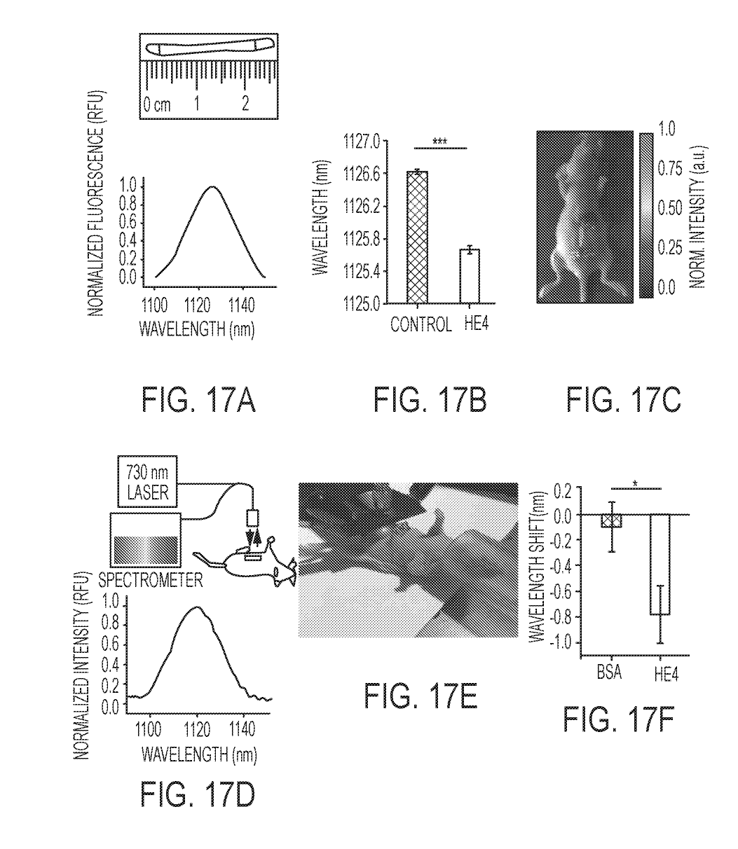

To assess the function of the nanosensor in vivo, a membrane-based device to implant the Ab-DNA-SWCNT complexes into live mice was developed. The passivated sensor was loaded into a semipermeable polyvinylidene fluoride (PVDF) membrane capillary with a molecular weight cut-off (MWCO) of 500 kDa. The material allowed excitation/emission of nanotubes through the membrane (FIG. 17A). It was estimated that the sensor complex was to be larger than the MWCO. Assuming an average diameter nanotube is 1.0 nm, the average length of nanotubes prepared via this method is 166 nm, and a 1:1 ssDNA to nanotube weight ratio from simulations, it was calculated that the DNA-SWCNT complex to be at least 1446 kDa (see Methods). The molecular weight of HE4 is approximately 25 kDa, allowing it to pass through the membrane. The response of the implantable sensor device was tested when it was immersed in 10% FBS, resulting in a 1 nm blue-shift upon exposure to 100 nM recombinant HE4 after 60 minutes, as compared to controls (FIG. 17B, p=7.2E⁻⁶).

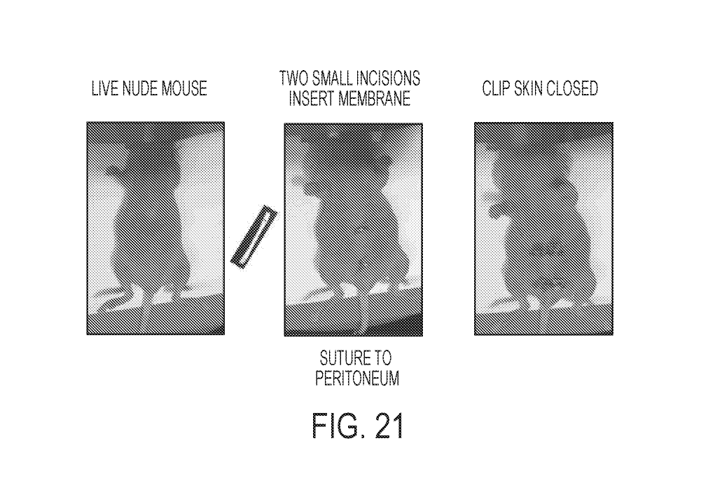

To investigate the functionality of the implantable sensor in vivo, its response to recombinant HE4 injected into the peritoneal cavity of live mice was investigated. Sensors were surgically implanted into healthy, 4-8 week female athymic nude mice (Envigo Hsd:Athymic Nude-Foxn1′) under anesthesia (FIG. 21; see Methods). The implant was sutured to the parietal peritoneum medially above the intestines, and the overlying skin was clipped closed. Placement of the implantable device and nanosensor emission from within the peritoneum were confirmed by whole-animal NIR imaging (FIG. 17C). Typical results revealed bright emission medially to the abdomen and no nanotube leakage from the membrane. Mice were then injected with 10 picomoles of HE4, an equal amount of BSA, or left untreated (N=3). The mice were allowed to become alert and ambulatory, exhibiting no adverse effects or signs of distress from surgery or the implanted device.

The implanted devices were interrogated non-invasively to assess the nanosensor response from injected HE4. To excite and collect light from the implant, a fiber optic probe-based system was developed to excite an area of approximately 0.8 cm² with a 730 nm laser (see Methods). Emission from the sensor was collected through the same fiber bundle which was coupled to a spectrometer/NIR array detector. Measurements were taken on mice re-anesthetized 60 minutes after HE4 injection. Spectra were acquired with 3-second integration time; 3 measurements were taken and averaged per mouse (FIGS. 17D-17E). Within HE4-injected mice, the sensors exhibited a 0.7 nm blue-shift as compared to controls (FIG. 17F, p=0.016)—almost identical to the magnitude observed in vitro upon interrogating with the same quantity of HE4 (0.6 nm blue-shift; FIG. 15B). Following sacrifice, the sensor device was removed and found to exhibit no compromise in structural integrity or function.

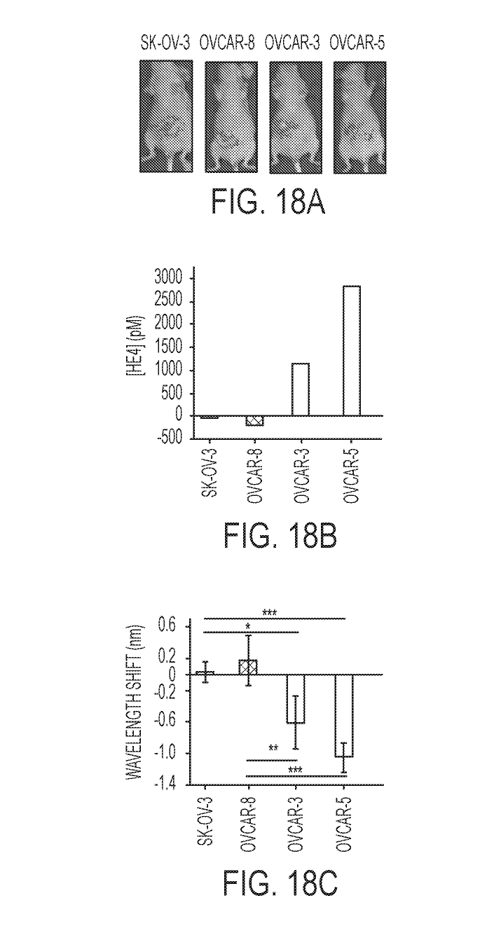

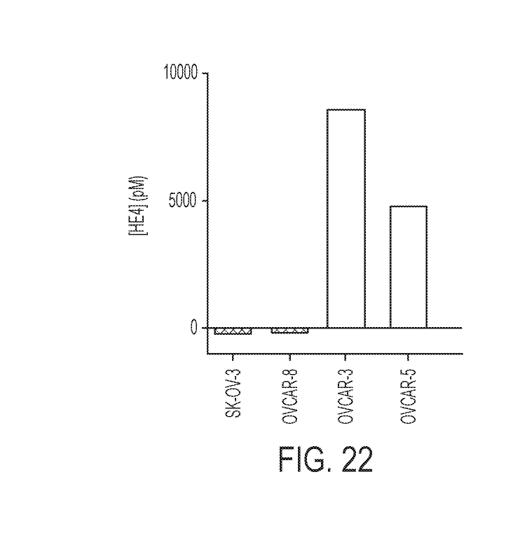

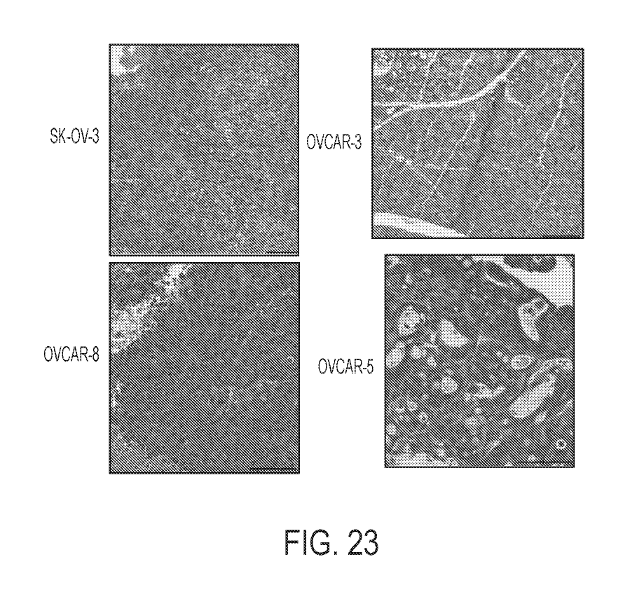

It was investigated whether the nanosensor could measure tumor-derived HE4 within orthotopic murine models of ovarian cancer. Four cohorts of athymic nude mice were injected intraperitoneally with approximately 10 million cells of four different luciferase-expressing cell lines: OVCAR-3, SK-OV-3, OVCAR-5, and OVCAR-8 (N=4 of each). The OVCAR-3 and OVCAR-5 cells express high levels of HE4, while SK-OV-3 and OVCAR-8 cells express low to negligible levels of HE4. These cell lines are thought to represent HGSC with the exception of SKOV3, which is likely not of HGSC origin. It was confirmed that HE4 expression via an enzyme-linked immunosorbent assay (ELISA) on conditioned cell culture media from each cell line (FIG. 22). Tumors were allowed to grow for approximately four weeks, after which in vivo bioluminescence imaging showed significant tumor burden in the mice (FIG. 18A). Mice exhibited distended, fluid-filled abdomens typical of ovarian cancer-associated ascites and solid tumor nodules in the peritoneal cavity. The presence of HE4 in OVCAR-3 and OVCAR-5 ascites and low concentrations or absence of HE4 in SK-OV-3 and OVCAR-8 ascites was confirmed via ELISA on ascites flushed from the peritoneal cavity with excess PBS (FIG. 18C). Tumor burden was further confirmed via H&E staining on resected tumor nodules (FIG. 23).

To measure HE4 in vivo using the nanosensor, the devices were implanted into tumor-bearing mice. Surgical procedures were performed as described heretofore on mice four weeks after tumor cell injection. Nanosensor emission was measured one hour after implantation using the fiber optic probe system via 3-second acquisitions. In mice bearing OVCAR-3 or OVCAR-5 cells, the sensors exhibited a 0.6 or 1.0 nm blue-shift, respectively, as compared to controls, while it exhibited a negligible change in SK-OV-3 or OVCAR-8 models (FIG. 18C). The mean emission wavelength of the sensor from each HE4 (−) mouse was significantly different from that of each HE4 (+) mouse. Given that OVCAR-5 cells express higher levels of HE4 than OVCAR-3 cells in vivo (FIG. 18B) and the sensor exhibited a larger blue-shift in OVCAR-5 bearing mice, this data further suggests the in vivo response of the sensor was quantitative.

This work describes the first in vivo quantification of a cancer biomarker using an optical sensor implant. The present Example provides a nanotube-based optical sensor for the ovarian cancer protein biomarker, such as HE4. It was found that the sensor can quantify HE4 in patient serum and ascites samples at relevant biomarker concentrations, potentiating future use as a rapid or point-of-care sensor. Development, characterization, and employment of an implantable sensor device to non-invasively detect tumor-derived HE4 in murine models of HGSC are described. Although many existing imaging modalities visualize tumors by binding protein targets, this work represents the first quantitative sampling of a local protein environment via an implantable nanosensor device. Thus, the first optical nanosensor-based in vivo detection of a cancer biomarker, directly correlated with disease state, in a robust, minimally-invasive manner is presented herein. The nanosensor complex is readily modifiable for the investigation of other proteins including biomarkers of other diseases. The current work also provides for in vivo optical biomarker detection in patients with risk factors for disease or for monitoring disease relapse following treatment of patients in remission.

### Methods

### Sensor Synthesis

The HE4 sensor complex was synthesized by probe-tip ultrasonication of as-prepared HiPCO single-walled carbon nanotubes (SWCNT) (Unidym; Sunnyvale, Calif.)) with amino-modified single-stranded DNA oligonucleotide with the sequence: 5′-(TAT)₆/AmMO/-3′ (Integrated DNA Technologies; Coralville, Iowa). Briefly, a 2:1 mass ratio of ssDNA to dried nanotubes was added to 1 mL 1× PBS and sonicated for 30 minutes at 40% of the maximum amplitude (˜13 Watts) (Sonics & Materials, Inc.; Newtown, Conn.). The suspensions were then ultracentrifuged (Sorvall Discovery 90SE; Waltham, Mass.) for 30 minutes at 280,000×g. The top 75% of the solution was removed for further processing, discarding the bottom 25% that contained unsuspended nanotubes and carbonaceous material. Amicon centrifugal filters with a 100 kDa MWCO were used (Millipore; Billerica, Mass.) to remove free ssDNA and to concentrate the samples, which were resuspended in 1× PBS. Absorbance spectra were obtained with a UV/Vis/nIR spectrophotometer (Jasco V-670; Tokyo, Japan) to determine sample concentration using the extinction coefficient Abs₆₃₀=0.036 L mg⁻¹ cm⁻¹.

The resulting DNA-SWCNT complex was then chemically conjugated via carbodiimide chemistry to goat polyclonal anti-HE4 IgG antibody (C-12, Santa Cruz Biotechnology; Dallas, Tex.) to form the Ab-DNA-SWCNT sensor construct. The carboxylic acids of the antibody were first activated with 1-ethyl-3-(3-dimethylainopropyl)carbodiimide (EDC) and N-hydroxysuccinimide (NHS) for 15 minutes. This reaction was quenched with 1.4 μL 2-mercaptoethanol. The activated antibody was added in an equimolar ratio to the ssDNA. Following two hours of incubation on ice, the conjugate was dialyzed against water with a 1 MDa MWCO filter (Float-A-Lyzer G2; Spectrum Labs; Irving, Tex.) at 4° C. for 48 hours with two buffer changes to remove unconjugated antibody and reaction reagents.

### Near-Infrared Spectroscopy and Imaging Experiments

Fluorescence emission spectra from antibody-conjugated and unconjugated nanotubes in solution were acquired using a home-built optical setup. This apparatus comprises a SuperK EXTREME supercontinuum tunable white light laser source (NKT Photonics; Birkerod, Denmark) with a VARIA tunable bandpass filter to modulate the output within the 500-825 nm range. A bandwidth of 20 nm was used. Alternatively, a 1 watt continuous-wave 730 nm laser source (Frankfurt; Friedrichsdorf, Germany) was used. The light path was shaped and fed into the back of an inverted IX-71 microscope (Olympus; Tokyo, Japan), passed through a 20× NIR objective (Olympus), to illuminate a 100 μL sample in a UV half-area 96 well plate (Corning; Corning, N.Y.). Emission was collected back through the 20× objective and passed through an 875 nm dichroic mirror (Semrock; Rochester, N.Y.). The light was f/# matched to the spectrometer using glass lenses and injected into an IsoPlane spectrograph (Princeton Instruments; Trenton, N.J.) with a 410 μm slit width. The emission dispersed using a 86 g/mm grating with 950 nm blaze wavelength. The spectral range was 930-1369 nm with a ˜0.7 nm resolution. The light was collected by a PIoNIR InGaAs 640×512 pixel array (Princeton Instruments). Single spectra were acquired using the 730 nm laser or the supercontinuum laser source with the variable bandpass filter centered at 730 nm. Excitation/emission plots, also dubbed photoluminescence (PL), plots, were compiled using the supercontinuum laser for excitation. Spectra were acquired between movements of the VARIA bandpass filter in 3 nm steps from 500-827 nm. A HL-3-CAL EXT halogen calibration light source (Ocean Optics; Dunedin, Fla.) was used to correct for wavelength-dependent features in the emission intensity arising from the excitation power, spectrometer, detector, and other optics. A Hg/Ne pencil-style calibration lamp (Newport; Irvine, Calif.) was used to calibrate spectrometer wavelength. Data were obtained from each well at multiple time points using custom LabView (National Instruments; Austin, Tex.) code. Background subtraction was performed using a well in the same plate with identical buffer conditions to the samples. Data was processed with custom MATLAB (MathWorks; Natick, Mass.) code, which applied spectral corrections as noted above, background subtraction, and data fitting with Lorentzian functions. All MATLAB code is available upon request.

Near-infrared fluorescence images and spectra were obtained from a hyperspectral microscope (Photon, Etc.; Montreal, Canada). Briefly, the setup consists of an inverted IX-71 microscope (Olympus). Experiments were performed with a continuous wave 2 watt 730 nm laser (Frankfurt) fed through a 100X oil immersion lens (Olympus). Nanotube samples immobilized on a glass surface were excited, and emission was collected through the objective. To obtain spectra, light was fed through a volume Bragg grating to obtain images in sequential 4 nm steps from 900-1400 nm (hyperspectral cubes). Light was collected using a 256×320 pixel InGaAs array.

Individual fluorescence spectra from implantable membranes in vivo and ex vivo were obtained using a home-built preclinical fiber-optic probe spectroscopy system. A continuous wave 1 watt 730 nm laser (Frankfurt) was injected into a bifurcated fiber optic reflection probe bundle. The bundle consisted of a 200 μm, 0.22 NA fiber optic cable for sample excitation located in the center of six 200 μm, 0.22 NA fibers for collection. Longpass filters were used to block emission below 1050 nm. The light was focused into a 303 mm focal length Czerny-Turner spectrograph (Shamrock 303i, Andor; Belfast, UK) with the slit width set at 410 μm. Light was dispersed by a 85 g/mm grating with 1350 nm blaze wavelength and collected with an iDus InGaAs camera (Andor). The spectra were processed to apply spectral corrections for non-linearity of the InGaAs detector response, background subtraction, and baseline subtraction via the use of OriginPro 9 software (Origin Lab; Northampton, Mass.) with a standard adjacent averaging smoothing method and a spline interpolation method. To quantify center wavelengths, spectra were fit to Voigt functions using custom MATLAB code.

Live animal NIR images were obtained using a pre-clinical NIR imaging apparatus consisting of a 2D InGaAs array and two 2 W 730 nm lasers (Photon, Etc.). The mouse was anesthetized with 1-3% isoflurane administered via nose cone during imaging. A 1100 nm long-pass filter was placed into the emission path to reduce autofluorescence. The background-subtracted NIR fluorescence image was overlaid on an image of the mouse taken under ambient visible light.

### In Vitro Sensor Characterization

Absorbance spectra of the Ab-DNA-SWCNT complex were obtained with a UV/Vis/NIR spectrophotometer as described above. Photoluminescence (PL) plots and individual spectra were obtained from the antibody-conjugated and unconjugated nanotubes using a home-built microscopy apparatus, as described herein. PL plots were obtained from the antibody-conjugated sensor and unconjugated control to determine the effect on each nanotube chirality of antibody conjugation. Individual spectra were obtained from samples using the 730 nm laser.

To test sensor response to HE4, the Ab-DNA-SWCNT complex was first incubated on ice with a 50× BSA:SWCNT ratio to passivate the nanotube surface. The passivated sensor complex was added to a 96 well plate at a nanotube concentration of 0.25 mg/L in a 100 μL total volume of PBS and 10% FBS (Gibco; New York, N.Y.). Recombinant human HE4 (G1u31-Phe124, RayBiotech; Norcross, Ga.) was added to the sensor complex in separate wells at concentrations of: 0 nM (baseline control), 1 pM, 10 pM, 100 pM, 1 nM, 10 nM, 50 nM, 100 nM, 250 nM, and 500 nM. Data were taken for up to 2 hours in 5 minute increments. Experiments were performed in triplicate.

To test sensor specificity, the Ab-DNA-SWCNT complex was first incubated with BSA on ice as above. Passivated sensor complexes were added to a 96 well plate at a nanotube concentration of 0.25 mg/L in a 100 μL total volume of PBS and 10% FBS. In triplicate, the following were added into wells: 500 nM recombinant human HE4, 500 nM recombinant human urokinase plasminogen activator (uPA)—a metastatic cancer biomarker (RayBiotech), 500 nM native human CA-125 of cellular origin (Cell Sciences; Canton, Mass.), 500 nm BSA (Sigma Aldrich; St. Louis, Mo.), or an additional 83% (for a total of 93%) FBS. To ensure specificity of the sensor construct, 500 nM recombinant human HE4 was added to DNA-SWCNT complexes without antibody as described above. Experiments were performed with the same time points as described herein.

### Ex Vivo Sensor Characterization

The non-passivated Ab-DNA-SWCNT sensor complex (10 μL) was added to a collagen-coated MatTek (Ashland, Mass.) glass-bottom dish for 30 seconds and removed, allowing the complexes to be deposited on the surface. Then, 90 μL of 1× PBS was added to the dish. A single broadband NIR fluorescence image was obtained in the 900-1400 nm range using the hyperspectral microscope described herein under 730 nm laser excitation. A continuous stack of emission wavelength-defined images (hyperspectral cube) was acquired with the volume Bragg grating in place, moving in 4 nm steps between 1150-1250 nm. Then, 10 μl (final concentration of 10 nM) recombinant HE4 was added to the PBS for 10 minutes before a second cube was acquired. Spectra from 50-100 individual nanotubes were processed as described herein, and the mean emission wavelength was calculated. A student's t-test was used to determine significance between the pre-HE4 and post-HE4 addition populations. A separate experiment was performed for an equal concentration of BSA to test specificity of the response of the sensor in this context.

The immobilized Ab-DNA-SWCNT complexes were interrogated with 10 μl of patient samples. Fluids from three separate patients with each condition were used: non-HGSC serum, HGSC serum, non-HGSC peritoneal fluid, and HGSC ascites. Each sample was obtained under MSKCC Institutional Review Board-approved protocols and informed consent was obtained. A student's t-test was performed to compare sensor shift for non-HGSC samples and HGSC samples. All patient samples (except one benign peritoneal fluid due to minimal volume obtained) were analyzed by ELISA to quantify HE4 (R&D Systems; Minneapolis, MN).

### Implantable Device Development

The Ab-DNA-SWCNT sensor complex was passivated by incubation on ice with BSA in a 50× BSA:SWCNT ratio for 30 minutes. FBS was then added to reach a 10% concentration. A semipermeable 500 kDa MWCO polyvinylidene fluoride (PVDF) KrosFlo dialysis membrane (Spectrum Labs; Rancho Dominguez, Calif.) ˜2 mm in diameter was cut to 2-3 cm long. A volume of 15-20 μL of 4 mg/L SWCNT (or 60-80 ng of the complex) was injected into the capillary. Both ends of the membrane were heat-sealed, leaving a ˜2 mm flap on each side.

### In Vitro Characterization of Implantable Device

The optical response of the Ab-DNA-SWCNT complex within the capillary device was tested by immersing the membrane in 1 mL 1× PBS and adding 100 nM recombinant HE4 to the solution. NIR emission of the nanotubes inside the membrane was obtained using the home-built microscopy setup as described herein. Spectra were obtained prior to HE4 addition and every 30 minutes thereafter. Background subtractions were performed with a blank membrane containing no nanotubes. Fluorescence measurements were taken in triplicate.

### Exogenous HE4 Detection In Vivo

All animal experiments were approved by the Institutional Animal Care and Use Committee at Memorial Sloan Kettering Cancer Center. Animal numbers were chosen to ensure repeatability while minimizing animal use. To test in vivo sensor functionality, 9 healthy, 4-8 week female athymic nude mice (Envigo Hsd: Athymic Nude-Foxn1^{nu}) were used to implant the membrane into the peritoneal cavity. Prior to implantation, NIR fluorescence spectra were acquired from the implant using the fiber optic probe spectroscopy apparatus described herein. Surgical implantation and fluorescence spectroscopy were performed under 1-3% isoflurane anesthesia, administered via nose cone. Between time-points, mice were alert and ambulatory, exhibiting no visible signs of pain or distress. Two ˜2 mm incisions were made in the skin and the below the parietal peritoneum, one ˜5 mm distal to the xiphoid process and one ˜2 cm distally of the first incision (FIG. 21). The membrane was inserted through the proximal incision through to the distal incision. The flaps at each end of the device were sutured to the parietal peritoneum using 5-0 Monocryl (poliglecaprone 25) absorbable sutures (Ethicon; Somerville, N.J.). The skin was clipped twice at each incision with 9 mm stainless steel AutoClips to close the incisions (MikRon Precision, Inc.; Gardena, Calif.). NIR fluorescence spectra from the sensor device were acquired using the probe-based system by pointing the fiber probe at the abdomen of the anesthetized mouse from a distance of 1-2 cm. Mice were then injected with 10 pmol HE4 or BSA in 100 μL PBS, or they were left uninjected as a second control (N=3). NIR spectra were then acquired 60 minutes following injection. Spectra were also collected at 2, 4, and 24 hours post-injection. Following 24 hours, mice were sacrificed and the implantable devices were removed. Spectra were again acquired from the device ex vivo. Spectra were processed as described herein.

### Murine Models

Luciferized cell lines OVCAR-3 [cultured in RPMI-1640+20% FBS+0.01 mg/mL insulin (Humulin R, Lilly; Indianapolis, Ind.)+100 μg/mL Primocin (InvivoGen; San Diego, Calif.)], SK-OV-3 [cultured in DMEM Low Glucose+10% FBS +Primocin] (ATCC), OVCAR-5, and OVCAR-8 [both cultured in RPMI-1640+1 mM sodium pyruvate+10% FBS+Primocin)] were grown at 37° C. under humid conditions. All culture reagents were from Gibco unless otherwise noted. Cells were passaged at 80-90% confluency approximately once weekly, and media was changed every 2-3 days. ELISA was performed to determine the presence of HE4 in conditioned culture media collected at ˜90% confluency (R&D Systems). Upon reaching near confluency, cells were trypsinized for 10 minutes at 37° C., complete media was added to deactivate trypsin, cells were centrifuged at 150× g for 7 minutes at 4° C., and pellets were resuspended in cold 1× PBS. Cells were counted using a Tali image-based cytometer (Invitrogen; Carlsbad, Calif.). Approximately 10 million cells in a 100 μL volume were injected intraperitoneally into 4-8 female athymic nude mice (N=4) (Envigo). Mice were housed under standard conditions and whole-animal bioluminescence imaging was performed twice weekly to monitor cell proliferation using an IVIS Spectrum In Vivo Imaging System (FIG. 18A) (Perkin Elmer; Waltham, Mass.) using standard firefly luciferase bioluminescence settings. Approximately 4-5 weeks following injection, maximal bioluminescence signal was obtained and most mice exhibited distended, fluid-filled abdomens typical of peritoneal ascites with some solid tumor nodules.

In Vivo Studies with Implantable Sensor Device

Prior to implantation, NIR spectra were acquired from the sensor devices using the probe-based spectroscopy system. Sensor devices were implanted into each mouse as described herein, with care taken to minimize loss of ascitic fluid. Spectra were obtained at 1, 2, 4, and 24 hours following implantation. After 24 hours, mice were sacrificed, the implant devices were removed, and spectra of the devices were acquired. The emission center wavelengths were compared to control uninjected mice to determine the magnitude of the shifts. Upon sacrificing the mice, ascitic fluid was removed directly from the peritoneal cavity of the mice with a needle and syringe or washed with up to 2 mL 1× PBS and removed. Solid tumor nodules were removed for histological analysis.

An enzyme-linked immunosorbent assays (ELISA) were performed using an HE4 kit (R&D Systems) to quantify HE4 mouse in ascites. Tumor tissues were fixed in 4% PFA, dehydrated, and paraffin embedded before 5μm sections were placed on glass slides. The paraffin was removed and slides were stained with haematoxylin and eosin (H&E) for basic histological analysis.

### Sensor Molecular Weight Calculation

The average molecular weight of the Ab-DNA-SWCNT complex was calculated given an average diameter nanotube of 1.0 nm in diameter (range: 0.8 nm-1.2 nm) (assumptions provided by the manufacturer). ((1.0 nm/0.245 nm)×3.1414×2 carbon atoms)=26 carbons around the circumference of the nanotube. Thus, there are 104 carbon atoms (4×26) for every 0.283 nm of nanotube length. For an average length of 166 nm (range: 100-1000 nm), the mass of a single narrowest-diameter nanotube is 723 kDa (166 nm/0.283 nm)(104 carbons)(12.01 amu of carbon). Given simulations, it was assumed a 1:1 weight ratio of ssDNA to SWCNT after sonication and purification to obtain a weight of 723 kDa×2=1446 kDa prior to antibody conjugation. The full pre-conjugation molecular weight range can be calculated by assuming a narrowest diameter of 0.8 nm and shortest length of 100 nm and a widest diameter of 1.2 nm and length of 1000 nm. This range, prior to ssDNA complexation, is 339 kDa-10524 kDa. Conjugation of an antibody will add approximately 150 kDa per antibody to the mass of the complex. Thus, a 500 kDa MWCO appears sufficient for retaining the Ab-DNA-SWCNT complex within the membrane.

1. A single-walled carbon nanotube (SWCNT) sensor, comprising a SWCNT;
a polymer associated with the SWCNT; and
an analyte-binding species.

2. The sensor of claim 1, wherein the analyte-binding species comprises a member selected from the group consisting of a peptide and a protein.

3. The sensor of claim 1, wherein the analyte-binding species comprises an antibody.

4. The sensor of claim 1, wherein the analyte-binding species is attached to the polymer via a functional group.

5. The sensor of claim 1, wherein the analyte-binding species binds a desired analyte; whereupon binding of the desired analyte to the analyte-binding species results in a detectable change in intrinsic fluorescence of the SWCNT.

6. The sensor of claim 5, wherein the desired analyte comprises a member selected from the group consisting of HE4, CA-125, mesothelin, cellular retinoic acid binding protein 2 (CRABP2), and YR L-4.

7. The sensor of claim 5, wherein the desired analyte comprises a member selected from the group consisting of uPA receptor (uPAR) YKL-40, prostate specific antigen (PSA), prostate specific membrane antigen (PSMA), carcinoembryonic antigen (CEA), and MUC1.

8. The sensor of claim 1, wherein the polymer is conjugated to the SWCNT via a linker.

9. The sensor of claim 1, wherein the polymer comprises a member selected from the group consisting of DNA, LNA, PNA, an amino-acid sequence, and a synthetic monomer,

10. The sensor of claim 1, wherein the polymer comprises DNA, and wherein the DNA is single-stranded DNA.

11-23. (canceled)

24. A system for in vivo detection of biomarkers and/or tumor cells in a subject, the system comprising:
an implantable device comprising the sensor of claim 1;
an excitation light source; and
a detector for detecting light emitted from the implantable device following excitation by the excitation light source.

25. The system of claim 24, wherein the implantable device comprises a biocompatible gel, microcapillary, filter, mesh, tubing, compartment/dialysis membrane, and/or other solid support on or in which the sensor is immobilized.

26. The system of claim 24, wherein the excitation light source emits near-infrared light or light having a wavelength greater than 700 nm.

27. (canceled)

28. The system of any one of claims 24, wherein, the implantable device is configured for implantation subcutaneously, intraperitoneally, and/or within the venous system.

29. The system of claim 28, wherein the implantable device is configured to be an intrauterine device (IUD).

30. The system of claim 28, wherein the implantable device is configured for delivery via injection.

31. The system of claim 28, wherein the implantable device is configured for implantation in the inferior vena cava.

32-36. (canceled)

37. A method of detecting an analyte of interest in a biological sample, the method comprising:
contacting the biological sample with the sensor of claim 1;
detecting an emission EMR of the sensor;
whereupon binding of the analyte to the analyte-binding species of the sensor a results in a detectable change in the emission EMR.

38-50. (canceled)

51. A kit for use in a laboratory setting, the kit comprising:
at least one container; and
the sensor of claim 1.

52. The sensor of claim 1, wherein the polymer comprises single-stranded DNA.
